(19) United States
(12) Patent Application Publication
(10) Pub. No.: US 2021/0299081 A1
(43) Pub. Date: Sep. 30, 2021

(54) SOLID CANNABINOID FORMULATION FOR ORAL ADMINISTRATION

(71) Applicant: Molecular Infusions, LLC, Franklin, MA (US)

(72) Inventors: Wenmin Yuan, Franklin, MA (US); Oren Levy, Brookline, MA (US); Tuna Yucel, Medford, MA (US)

(21) Appl. No.: 17/210,810

(22) Filed: Mar. 24, 2021

Related U.S. Application Data
(60) Provisional application No. 62/994,601, filed on Mar. 25, 2020.

(51) Int. Cl. A61K 31/352; A61K 31/05; A61K 9/16; A61K 9/107; A61K 47/44; A61K 9/00; A61K 9/46; A61K 9/20; A61K 9/48; A61K 9/28

(52) U.S. Cl. CPC A61K 31/352; A61K 9/2846; A61K 9/1623; A61K 9/1075; A61K 9/1694; A61K 47/44; A61K 9/0095; A61K 9/0007; A61K 9/2031; A61K 9/2013; A61K 9/2018; A61K 9/4825; A61K 9/4858; A61K 9/4866; A61K 31/05

(57) ABSTRACT

The invention provides solid, self-emulsifying compositions for oral administration. The compositions can comprise a population of particles comprising a cannabinoid or a terpene, a surfactant, and a solid carrier. The invention also encompasses methods for the preparation and use thereof.

### RELATED APPLICATION

This application claims the benefit of U.S. Provisional Application Ser. No. 62/994,601 filed Mar. 25, 2020. The entire contents of this application are incorporated by reference herein.

### BACKGROUND OF THE INVENTION

Cannabinoids are a class of active compounds derived from the Cannabis sativa, Cannabis indica, or cannabis hybrid plants commonly known as marijuana. The most notable cannabinoid is the phytocannabinoid tetrahydrocannabinol (THC), the primary psychoactive compound in cannabis. Delta-9-tetrahydrocannabinol (Δ9-THC) and delta-8-tetrahydrocannabinol (Δ8-THC) mimic the actions of anandamide and 2-arachidonoylglycerol neurotransmitters produced naturally in the body. These cannabinoids produce the effects associated with cannabis by binding to the CB1 cannabinoid receptors in the brain. Cannabidiol (CBD) is another major constituent of the cannabis plant. Other cannabinoids include Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), Tetrahydrocannabinolic acid (THCA), cannabinol (CBN), Cannabidiolic Acid (CBDA), Delta-9-tetrahydrocannabiphorol (THCP), Delta-9-tetrahydrocannabutol (THCB), cannabidiphorol (CBDP), and cannabidibutol (CBDB).

Through state-regulated markets, cannabis consumption is now legal in some form in most U.S. states. With increasing legalization comes a growing interest towards development of safe and efficacious cannabinoid products. The oral delivery route remains the preferred route of drug administration due to high patient compliance and ease of administration [Pharmaceutics. 2018 Dec; 10(4): 263]. However, most cannabinoids, such as Δ⁹-tetrahydrocannabinol (Δ9-THC) and cannabidiol (CBD) are highly lipophilic and poorly water soluble. As a result, current pharmaceutical oral cannabinoid products typically display poor oral pharmacokinetics (PK, see Table 1 below), such as low and highly variable bioavailability, along with undesirable food effects. These poor characteristics generally result in unpredictable efficacy after oral administration (e.g., variable onset time and/or peak of observed effects) together with potential safety concerns, e.g., overdosing especially in geriatric use. Therefore, there is a clear and urgent need to develop oral cannabinoid formulations to address these challenges.

TABLE 1
Current pharmaceutical oral cannabinoid products:

| Product | Dosage form/ | | Absorption | | |
|---|---|---|---|---|---|
| (sponsor) | Formulation | Bioavailability | (Tmax) | Variability | Food effects |
| Marinol | Oral capsule; | 4-20%¹ | 1-2.5 h | High inter- and | Food increased AUC |
| (Abbvie) | solution in | | (median, | intra-subject | and delayed Tmax |
| | sesame oil | | fasted) | variability | |
| Syndros | Oral solution in | ca. 20% higher | 1 h | 60% reduction in | Food increased AUC |
| (Insys) | EtOH, PG, PEG, | than Marinol | (median, | intra-subject | and delayed Tmax |
| | Water | | fasted) | variability vs. | |
| | | | | Marinol | |
| Namisol | Oral tablet; | N/R | 0.7-1 h | Highly variable but | Not reported |
| (Echo) | Alitra technology | | (mean) | less variable vs. | |
| | | | | Marinol | |
| Sativex | Oromucosal | Similar to | 1.5-3.3 h | High inter- and | Food increased AUC |
| (GWPharma) | spray; solution in | Marinol | (mean, | intra-subject | |
| | EtOH, PG | | fasted) | variability | |
| Epidiolex | Oral solution in | Not reported | 2.5-5 h | Higher variability in | Food increased AUC |
| (GWPharma) | sesame oil and | | | fasted vs. fed state | (4-fold) and Cmax |
| | EtOH | | | in healthy subjects | (5-fold) |

In order to enhance the oral PK of cannabinoids, liquid cannabinoid pre-concentrates for oral administration have been described (PCT Publication No. WO2018152334; PCT Application No. PCT/US2018/018382). These compositions simultaneously “self-emulsify” into nanoparticle dispersions upon dilution in the GI tract when administered as an oral capsule. Self-emulsification into nanoparticles led to enhanced aqueous solubilization of cannabinoids, resulting in improved self-reported effects (e.g., faster onset and peak of effects, stronger overall effects) and symptomatic relief (e.g., relief of pain) in consumers versus a MARINOL® comparator. Further, these pre-concentrates could conveniently be consumed as beverage additives due to their compatibility with most commercially available beverages. These liquid oral cannabinoid compositions included one or more surfactant(s) with a high Hydrophilic to Lipophilic Balance or “HLB value.” However, the high viscosity of most hydrophilic surfactants, along with the semi-solid (in the case of THC) or crystalline (in the case of CBD) nature of major cannabinoids can result in high formulation viscosity. Subsequently, a co-solvent was introduced into the formulation to enhance its dissolution kinetics and obtain fast dissolving oral capsule and beverage additive formulations (PCT Publication No. WO2019036243; PCT Application No. PCT/US2018/045714). Along with enhanced dissolution kinetics, consumption of co-solvent-containing formulas generally resulted in further enhanced self-reported psychoactive effects and symptomatic relief. Another potential benefit of co-solvent added formulations was enhancing the cannabinoid dose homogeneity in the media that they are dispersed (GI tract or beverage of choice) and thereby potentially enhancing product safety.

Despite their desirable in vitro (e.g., dissolution, dispersion) and in vivo properties (e.g., enhanced self-reported effects in consumers), the use of a co-solvent in these compositions can also result in certain limitations, such as: container closure compatibility, potential incompatibility with semi-permeable or poorly sealed packaging, as well as potential of leaching, especially for multi-use products; potential co-solvent-concomitant drug interactions, along with populations with sensitivity to certain co-solvents; incompatibility with solid formats (e.g., powder, tablets, hard capsules); and potentially more complicated filling, storage and/or shipping requirements versus solid formulations.

There remains a need in the art for improved cannabinoid compositions for oral administration.

### SUMMARY OF THE INVENTION

The present invention is directed to solid, self-emulsifying compositions for oral administration that overcome the limitations of their liquid counterparts, while retaining their desirable in vitro (e.g., dissolution and dispersion) and in vivo (self-report effects and symptom relief in consumers) effects.

The invention includes a self-emulsifying, solid composition comprising a population of particles, wherein each particle comprises: i) at least one of a cannabinoid and a terpene, ii) at least one surfactant having an HLB value greater than 10, and iii) a solid carrier in powder or granular form, wherein the solid carrier is a water-soluble solid carrier or a water-insoluble solid carrier;

wherein when the solid carrier is a water-soluble solid carrier, then the surfactant and the cannabinoid or the terpene are present on the surface of the solid carrier, or the water-soluble carrier, surfactant, and the cannabinoid or the terpene form an amorphous matrix; and

wherein when the solid carrier is a water-insoluble solid carrier, then the surfactant and the cannabinoid or the terpene are present on the surface of the solid carrier.

In certain specific embodiments, the solid carrier is a water-soluble solid carrier and the surfactant and the cannabinoid or the terpene are present on the surface of the solid carrier (thus having a core-shell structure). In yet additional aspects, the solid carrier is a water-soluble solid carrier and the water-soluble carrier, surfactant, and the cannabinoid or the terpene form an amorphous matrix. In yet further aspects, the solid carrier is a water-insoluble solid carrier and the surfactant and the cannabinoid or the terpene are present on the surface of the solid carrier (thus having a core-shell structure).

In yet further specific aspects, the invention is directed to a self-emulsifying, solid composition comprising a population of particles, wherein each particle comprises: i) at least one of a cannabinoid and a terpene, ii) at least one surfactant having an HLB value greater than 10, and iii) mannitol in powder or granular form, wherein the surfactant and the cannabinoid or the terpene are present on the surface of the solid carrier. In yet further specific aspects, the invention is directed to a self-emulsifying, solid composition comprising a population of particles, wherein each particle comprises: i) at least one of a cannabinoid and a terpene, ii) D-α-tocopherol polyethylene glycol 1000 succinate (TPGS), and iii) mannitol in powder or granular form, wherein the surfactant and the cannabinoid or the terpene are present on the surface of the solid carrier. In yet additional aspects, the invention is directed to a self-emulsifying, solid composition comprising a population of particles, wherein each particle comprises: i) at least one of a cannabinoid and a terpene, ii) D-α-tocopherol polyethylene glycol 1000 succinate (TPGS) and polysorbate 80 (TWEEN 80), and iii) mannitol in powder or granular form, wherein the surfactant and the cannabinoid or the terpene are present on the surface of the solid carrier.

Other specific embodiments are directed to a self-emulsifying, solid composition comprising a population of particles, wherein each particle comprises: i) at least one of a cannabinoid and a terpene, ii) at least one surfactant having an HLB value greater than 10, and iii) one or more water-soluble solid carrier(s) in powder or granular form, wherein the solid carrier, surfactant and the cannabinoid or the terpene form an amorphous matrix (e.g., the surfactant and the cannabinoid are present homogeneously throughout the particle cross-section forming an amorphous mixture with the solid carrier). In certain specific embodiments, the water-soluble solid carrier is mannitol.

The compositions described herein can form self-assembled nanometer- and/or micrometer-sized particles upon addition to an aqueous medium. In certain aspects, the self-assembled nanoparticles are about 10 to about 800 nanometers (nm) in diameter.

The invention also includes a method for the preparation of a self-emulsifying, solid composition comprising a population of particles as described herein, wherein the surfactant and the cannabinoid or the terpene are present on the surface of the solid carrier, wherein the method comprises:
a. dissolving the cannabinoid or the terpene in a solvent mixture to form a first solution, wherein the solvent mixture comprises a volatile solvent and at least one surfactant;
b. mixing the solid carrier with the first solution to form a mixture, wherein the solid carrier is insoluble in the first solution;
c. evaporating the solvent from the mixture; and
d. collecting the particles.

In certain aspects, the solid carrier is a water-soluble solid carrier (including, but not limited to, mannitol). In yet additional aspects, the solid carrier is a water-insoluble solid carrier.

In yet further aspects, the invention is directed to a method for the preparation of a self-emulsifying, solid composition described herein, wherein the solid carrier, surfactant and the cannabinoid or the terpene form an amorphous solid, wherein the method comprises:
a. dissolving the cannabinoid or the terpene in a solvent mixture to form a first solution, wherein the solvent mixture comprises a volatile solvent and at least one surfactant;
b. mixing the water-soluble, solid carrier with the first solution to form a second solution, wherein the water-soluble, solid carrier is soluble in the first solution;
c. drying the second solution; for example, by hot melt extrusion, spray drying, or lyophilization/freeze drying to form an amorphous solid;
d. breaking the amorphous solid into particles; and
e. collecting the particles.

The invention also includes a method for the preparation of a self-emulsifying, solid composition as described herein, wherein the solid carrier, surfactant and the cannabinoid or the terpene form an amorphous solid, the method comprising:
a. mixing the cannabinoid or the terpene with the water-soluble solid carrier (for example, in the absence of a solvent) and forming an amorphous matrix by hot melt extrusion; and
b. breaking the amorphous solid into particles; and
c. collecting the particles.

Also encompassed is a method of dispersing or dissolving a cannabinoid or a terpene in an aqueous medium, the method comprising adding the self-emulsifying, solid composition described herein to an aqueous medium. The aqueous medium can, for example, be a beverage such as drinking water. The aqueous medium can also, for example, be gastric fluid and/or intestinal fluid.

The invention additionally includes a method of orally administering a cannabinoid or a terpene to a subject comprising oral administration of the self-emulsifying solid composition described herein.

In certain specific aspects, the water-soluble solid carrier is a sugar or a sugar alcohol, including, but not limited to, mannitol.

In further aspects, the average diameter of the solid carrier is less than about 3000 micrometers (um). In additional aspects, the average diameter of the solid carrier is greater than about 10 micrometers.

In some embodiments, the surfactant is selected from the group consisting of D-α-tocopherol polyethylene glycol 1000 succinate (TPGS), polysorbates, PEG-castor oils, phospholipid, lauroyl-L-carnitine, and poloxamers. In certain specific embodiments, the surfactant is TPGS. In additional aspects, the surfactant is a mixture of TGPS and Tween 80.

The amount of the solid carrier can, for example, be between about 50 to about 99% w/w.

In yet further aspects, the cannabinoid is selected from the group consisting of CBD, Δ9-tetrahydrocannabinol (Δ9-THC), Δ8-tetrahydrocannabinol (Δ8-THC), CBG, THCV, CBN, CBC, and CBDV, or a combination thereof. In additional embodiments, the cannabinoid is Δ9-THC or CBD, or a combination thereof.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic representation of cannabinoid self-emulsification solid formulation.

FIG. 2 is a schematic showing the preparation of solid, self-emulsifying oral dosage forms: direct coating of solid powder with Solution A.

FIGS. 3A, 3B and 3C are photographs of THC oral solid powders direct coating preparation by mixing solution A (containing THC) with different physical forms of mannitol (crystal, spray-dried, and granulated mannitol, respectively). Use of spray-dried mannitol resulted in better powder flowability versus crystal mannitol, and higher drug loading versus granulated mannitol.

FIGS. 4A and 4B are photographs showing absorption of Solution A on spray-dried mannitol and granulated mannitol before evaporation of ethanol. Spray-dried mannitol absorbed a higher content of Solution A as compared to granulated mannitol. As shown in FIG. 4A, spray dried mannitol could fully absorb Solution A resulting in a fine powder. In contrast, FIG. 4B shows that, at the same ratio of mannitol: Solution A, there was visible residual liquid when using granulated mannitol.

FIG. 5 is a photograph showing the aqueous dispersion properties of THC Solution A (left) vs. THC powder (right). Dispersion time was >5 min for THC Solution A (self-emulsifying liquid), while THC powder (self-emulsifying solid) dissolved within 10 seconds at the same target THC concentration (0.5 mg/ml).

FIGS. 6A, 6B, 6C and 6D show DLS particle size distribution of aqueous dispersions of THC and CBD powder formulations with and without added exogeneous oils. All formulations were dissolved in deionized (DI) water at 0.1 mg/ml cannabinoid concentration. For THC dispersions, exogeneous oils increased particle size, while the opposite was observed for CBD dispersions.

FIG. 7 is a photograph showing aqueous dispersions of CBD powder formulations. These formulations were prepared using different exogenous oils at varying oil to CBD ratios; the oil to CBD (oil: CBD) ratio of the three vials on the right of the photograph is double (2×) that of the three vials in the middle of the photograph. Aqueous dispersions were prepared in DI water at 0.1 mg/mL CBD. Note the decrease in apparent turbidity when exogeneous oil 1 was added at the 2× mass ratio (the circled vial).

FIG. 8 is a graph showing API percent concentration change for different powder formulations in accelerated stability study (60° C. incubation). The powder with antioxidant (TPGS) are more stable than without TPGS.

### DETAILED DESCRIPTION OF THE INVENTION

A description of preferred embodiments of the invention follows.

As used herein, the words “a” and “an” are meant to include one or more unless otherwise specified. For example, the term “a cannabinoid” encompasses both a single cannabinoid and a combination of two or more cannabinoids. The term “a surfactant” encompasses both a single surfactant and a combination of two or more surfactants.

Unless otherwise indicated, all numbers expressing reaction conditions, quantities of ingredients, and so forth, as used in this specification and the claims are to be understood as being modified in all instances by the term “about.”

As used herein, when a range is set forth as “between” two values, it is understood that the range is inclusive of the end values.

As used herein, the terms “treat”, “treating” or “treatment” means to alleviate, reduce or abrogate one or more symptoms or characteristics of a disease, disorder or event, cause a desired biological effect, and/or may be curative, palliative, prophylactic or slow the progression of the disease or disorder. Treatment can include achieving a psychoactive effect in an individual.

The term “effective amount” means an amount of active ingredient(s) that will result in a desired effect or result, including causing a psychoactive effect in an individual. The term “therapeutically effective amount” means an amount of active ingredient(s) that will elicit a desired biological or pharmacological response, e.g., effective to prevent, alleviate, or ameliorate symptoms (e.g., reducing or eliminating irritation and/or coughing and/or respiratory tract irritation), treat a disease or disorder (e.g., nausea); or cause a psychoactive effect in the individual. The compositions described herein can comprising an effective or therapeutically effective amount of a cannabinoid and/or a terpene.

The term “patient” or “subject” means an animal, including mammals, non-human animals, and especially humans. In one embodiment, the patient or subject is a human. In another embodiment, the patient or subject is a human male. In another embodiment, the patient or subject is a human female. The patient can be a healthy individual or an individual in need of medical treatment. In particular, the term “patient” is intended to include individuals that can medically benefit from the administration of a cannabinoid as well as individuals who can benefit recreationally.

As used herein, “surfactant” refers to synthetic and naturally occurring amphiphilic molecules that have hydrophobic portion(s) and hydrophilic portion(s). Due to their amphiphilic (amphipathic) nature, surfactants typically can reduce the surface tension between two immiscible liquids, for example, the oil and water phases in an emulsion, stabilizing the emulsion. Surfactants can be characterized based on their relative hydrophobicity and/or hydrophilicity. For example, relatively lipophilic surfactants are more soluble in fats, oils and waxes, and typically have HLB values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10 or about 10. As discussed above, self-emulsifying compositions comprising cannabinoids and surfactants have been described in WO2018152334 and WO2019036243, the contents of which are expressly incorporated by reference herein.

“Pharmaceutically acceptable salts,” or “salts,” include the salt of a cannabinoid (including, for example, a cannabinoid prodrug or a cannabinoid synthetic analog that includes a basic group) suitable for administration to a mammal, including those prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric, galacturonic, hydrochloric, hydrobromic, sulfuric, nitric, and phosphoric acids.

Described herein are solid, self-emulsifying compositions for oral administration that comprise:
(1) at least one cannabis-derived pharmaceutically active ingredient;
(2) at least one surfactant with a high HLB value; and
(3) at least one solid carrier in powder or granular form.

The solid carrier can be a water-soluble solid carrier or water-insoluble solid carrier. The compositions can include one or more additional ingredients including, but not limited to, one or more exogeneous oils, such as medium-chain triglycerides (MCT) oil, one or more antioxidants, such as tocopherols, ascorbates, one or more colorants, and one or more flavorings. In certain aspects, the solid carrier is a water-soluble solid carrier or a water-insoluble carrier and the formed particles have a core-shell structure, wherein the solid carrier forms a core and the core is coated by the surfactant and the cannabinoid and/or the terpene (forming the shell around the core). In certain additional aspects, the solid carrier is a water-soluble solid carrier and the formed particles form an amorphous matrix structure (e.g., the surfactant and the cannabinoid are present homogeneously throughout the particle cross-section forming an amorphous mixture with the solid carrier).

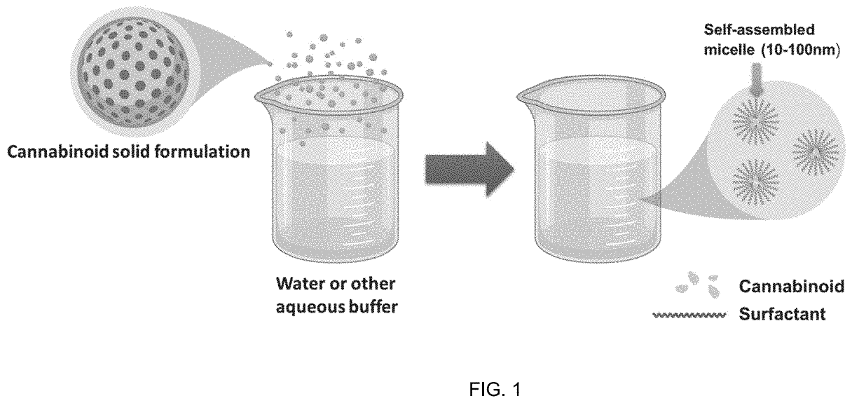

Unlike liquid self-emulsifying formulations that rely on a co-solvent to enhance dissolution kinetics, the solid formulations described herein utilize a micron-sized solid carrier to significantly increase the surface area and facilitate the self-emulsification process (see, e.g., FIG. 1). As such, the compositions can be substantially free of co-solvents and can be manufactured into solid formats including, but not limited to, powders, pellets and granules, and then further incorporated into tablets and capsules. As described above, additional benefits of the absence of a co-solvent include potential improved safety and better container/closure compatibility.

As described above, the solid composition comprises:

i. at least one cannabis-derived pharmaceutically active ingredient, such as cannabinoid(s) and/or terpene(s) (non-limiting examples of cannabis-derived pharmaceutically active ingredients are THC, CBD, terpenoids, flavonoids, polyphenols, or a combination thereof); the cannabis-derived pharmaceutically active ingredient can be present in amount of about 0.01 to about 10% w/w, for example; or a pharmaceutically acceptable derivative thereof;

ii. at least one high HLB surfactant (e.g., D-α-Tocopherol polyethylene glycol 1000 succinate (TPGS), polysorbates, PEG-castor oils, phospholipid, lauroyl-L-carnitine, poloxamers); the high HLB surfactant can be present in an amount from about 0.1 to about 50% w/w, for example; and

iii. at least one solid carrier; the solid carrier can be a water-soluble carrier (e.g., sucrose, fructose, maltose, glucose, lactose, galactose, mannitol, sorbitol, xylitol, starch, dextrin, maltodextrin, hydroxypropyl-methylcellulose, sodium carboxymethyl starch) or the solid carrier can be a water-insoluble carrier; and wherein the solid carrier can be present in an amount from about 50 to about 99% w/w, for example.

The invention encompasses a self-emulsifying, solid composition comprising a population of particles, wherein each particle comprises: i) at least one of a cannabinoid and a terpene, ii) at least one surfactant having an HLB value greater than 10, and iii) a solid carrier, for example, a water-soluble carrier or a water-insoluble solid carrier, in powder or granular form, wherein the surfactant and the cannabinoid or the terpene are present on the surface of the solid carrier. In additional aspects, the invention encompasses a self-emulsifying, solid composition comprising a population of particles, wherein each particle comprises: i) at least one of a cannabinoid and a terpene, ii) at least one surfactant having an HLB value greater than 10, and iii) a solid carrier, for example, a water-soluble carrier, in powder or granular form, wherein the surfactant and the cannabinoid or the terpene form an amorphous matrix or an amorphous solid. The invention also includes methods for the preparation and use of the self-emulsifying, solid compositions. In certain preferred aspects, the water-soluble solid carrier is mannitol. In additional aspects, the surfactant is TPGS.

The term “solid carrier,” as used herein, denotes a pharmaceutically acceptable solid material, for example, in solid or granular form. The solid carrier(s) used in the compositions described herein is one that does not adversely interact with the cannabinoid or the terpene in the composition. In certain aspects, the solid carrier has a high surface area. The solid carrier can be micron-sized. For example, the average diameter of the solid carrier can be less than about 5000 micrometers (μm), less than about 3000 micrometers, less than about 2000 micrometers, less than about 1000 micrometers, less than 750 micrometers, or less than about 500 micrometers. In yet additional aspects, the average diameter of the solid carrier is greater than about 10 micrometers, greater than about 30 micrometers, greater than about 50 micrometers, or greater than about 75 micrometers. In some aspects, the average diameter of the solid carrier is between about 10 and about 5000 micrometers, between about 10 and about 3000 micrometers, between about 10 and about 2000 micrometers, between about 10 and about 1000 micrometers, between about 10 and about 750 micrometers, or between about 10 and about 500 micrometers. In additional embodiments, average diameter of the solid carrier is between about 75 and about 5000 micrometers, between about 75 and about 3000 micrometers, between about 75 and about 2000 micrometers, between about 75 and about 1000 micrometers, between about 75 and about 750 micrometers, or between about 75 and about 500 micrometers. In certain aspects, the solid carrier has a high specific surface area, for example, the solid particle can have an average diameter of about 75 to about 500 micrometers and a specific surface area of at least about 0.1 m²/g, at least about 0.2 m²/g, or at least about 0.3 mg^{t}/g; or a specific surface area in a range between about 0.1 m²/g to about 5 m²/g, or between about 0.1 m²/g and about 3 m²/g. In yet further aspects, the solid carrier is mannitol (for example, granulated or spray-dried mannitol) having an average diameter between about 175 um and 550 um, and the surface area is at least about 0.3 m²/g, or between about 0.3 m² and 1.5 m²/g. In yet additional embodiments, the solid carrier is granulated mannitol, the average diameter of the carrier is between about 300 μm and 500 μm, and the surface area is between about 0.35 to about 0.40 m²/g, or about 0.37 to about 0.39 m²/g. In yet further aspects, the solid carrier is spray-dried mannitol, the average diameter of the solid carrier is between about 175 μm and 250 μm (e.g., about 200 μm), and the surface are of the solid carrier is between about 1.0 m²/g and 1.5 m²/g, or about 1.2 m²/g. The surface area of the solid carrier can be measured using standard procedures, such as low-temperature nitrogen adsorption, based on the Brunauer, Emmett, and Teller (BET) method.

As described above, the solid carrier can be in powder or granular form. The solid carrier can be a water-soluble solid carrier such as a sugar or a sugar alcohol. Non-limiting examples of sugars and sugar alcohols are sucrose, fructose, maltose, glucose, lactose, galactose, mannitol, sorbitol, and xylitol. In additional aspects, the solid carrier is selected from the group consisting of starch, dextrin, maltodextrin, hydroxypropyl-methylcellulose, and sodium carboxymethyl starch. In preferred aspects, the solid carrier is a sugar alcohol. More preferably, the sugar alcohol is mannitol. The mannitol solid carrier can be crystalline, granulated, or spray-dried. In certain embodiments, the mannitol is spray-dried.

In certain aspects, the solid carrier is in powder or granular form and is a water-insoluble solid carrier. Non-limiting examples of water-insoluble solid carriers are microcrystalline cellulose, silicon dioxide, magnesium aluminometasilicate, silica, sucrose monopalmitate, and calcium silicate.

In yet additional aspects, the composition comprises two populations of particles: wherein the composition comprises a first population of particles and a second population of particles wherein:
a. each particle of the first population comprises: i) at least one of a cannabinoid and a terpene, ii) at least one surfactant having an HLB value greater than 10, and iii) a water-soluble solid carrier in powder or granular form; and
b. each particle of the second population comprises i) at least one of a cannabinoid and a terpene, ii) at least one surfactant having an HLB value greater than 10, and iii) a water-insoluble solid carrier in powder or granular form.

The amount of the solid carrier (the water-soluble solid carrier or the water-insoluble solid carrier, or combination thereof) in the composition can be from about 50 to about 99% w/w.

As discussed above, the composition comprises a surfactant having an HLB value greater than 10. In other aspects, the surfactant has an HLB value of 11 or more, or an HLB value of 12 or more. Non-limiting examples of surfactants are PEG 15 hydroxystearate (Solutol HS15), polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40), polyethylene-polypropylene glycol (poloxamer 124), PEG 8 caprylic/capric glycerides (Labrasol), lauroyl macrogol 32 glycerides (GELUCIRE® 44/14), D-α-Tocopherol polyethylene glycol 1000 succinate (TPGS), polyethylene-polypropylene glycol (poloxamer 188), polyethylene-polypropylene glycol (poloxamer 407), hydroxypropyl methylcellulose (HPMC), carboxymethyl cellulose (CMC), polyethylene-polypropylene glycol (poloxamer 124), polyethylene glycol sorbitan monolaurate (polysorbate 20, TWEEN 20), polyethylene glycol sorbitan monopalmitate (polysorbate 40, TWEEN 40), polyethylene glycol sorbitan monostearate (polysorbate 60, TWEEN 60), polyethylene glycol sorbitan tristearate (polysorbate 65, TWEEN 65), polyethylene glycol sorbitan monooleate (polysorbate 80, TWEEN 80), polyethylene glycol sorbitan trioleate (polysorbate 85, TWEEN 85), polyethylene glycol sorbitan hexaoleate, polyethylene glycol sorbitan tetraoleate, sucrose laurate, sucrose palmitate, sucrose stearate, quillaia/quillaja saponins, quillaia extract, PEG 8 stearate, PEG 40 stearate, or a combination thereof. In certain embodiments, the surfactant is selected from the group consisting of D-α-tocopherol polyethylene glycol 1000 succinate (TPGS), polysorbates, PEG-castor oils, phospholipid, lauroyl-L-carnitine, and poloxamers. In certain preferred aspects, the surfactant is a mixture of TPGS and polysorbate 80.

In certain aspects, the surfactant has an HLB value greater than 10 as described herein and is an antioxidant. An example of such an antioxidant surfactant is TPGS, soy phosphatidylcholine (L-α-phosphatidylcholine (soy)), and egg phosphatidylcholine (L-α-phosphatidylcholine (egg)). A preferred an antioxidant surfactant is TPGS.

The amount of high HLB surfactant in the composition can be between about 0.1 and about 50% w/w.

The composition described herein can further comprise a low HLB surfactant (in addition to the surfactant that has an HLB value greater than 10). Non-limiting examples of a low HLB surfactant are Tween 61, sorbitan monolaurate (Span 20), sorbitan monopalmitate (Span 40), sorbitan monostearate (Span 60), sorbitan tristearate (Span 65), sorbitane monooleate (Span 80), and sorbitan trioleate (Span 85), PEG 300 oleic glycerides (Labrafil M 1944), diethylene glycol monoethyl ether (Transcutol), propylene glycol laurate (Lauroglycol FCC), lecithin, soy phosphatidylcholine (L-α-phosphatidylcholine (Soy)), egg phosphatidylcholine (L-α-phosphatidylcholine (egg)), caseinates, and acacia gum.

In certain aspects, the composition comprises a cannabinoid. In additional aspects, the composition comprises a terpene. In yet further aspects, the composition comprises a cannabinoid and a terpene. Cannabinoids include, but are not limited to, those described in the section below. Preferred cannabinoids include CBD, Δ9-tetrahydrocannabinol (Δ9-THC), Δ8-tetrahydrocannabinol (Δ8-THC), THCA, THCB, THCP, THCV, CBD, CBDA, CBDB, CBDP, CBDV, CBDL, CBC, CBCA, CBCV, CBCN, CBV, CBG, CBGA, CBGV, CBN, CBL, and CBE, or a combination thereof. In further preferred aspects, the composition comprises Δ9-THC or CBD, or a combination thereof. Terpenes include, but are not limited to, those described in the section below. In certain aspects, the terpene is selected from the group consisting of alpha-bisabolol, alpha-phellandrene, alpha-pinene, alpha-terpinene, alpha-terpineol, beta-caryophyllene, α-pinene, beta-pinene, borneol, cadinene, camphene, camphor, carvacrol, β-caryophyllene, caryophyllene acetate, caryophyllene oxide, cedrane, citral, citronellol, dextro carvone, dextro fenchone, eucalyptol (1,8-cineole), eugenol, farnesene, gamma-3-carene, gamma-terpinene, geraniol, geranyl acetate, guaiene, humulene, isopulegol, limonene, linalool, linalyl acetate, menthol, myrcene, b-myrcene, nerol, nerolidol, ocimene, ocimene, p-cymene, phytol, pulegone, terpineol, terpinen-4-ol, terpinolele, terpinolene, thymol, valencene, valencene, 1-menthol, or a combination thereof.

The amount of the cannabinoid or terpene in the composition can be between about 0.01 and about 20% w/w, or between about 0.01 and about 10% w/w, or between about 0.01 and about 7% w/w. In additional aspects, the composition has a total cannabinoid concentration between about 0.1 to about 200 mg/g.

The compositions can optionally further comprise, one or more exogenous oils (such as, almond oil, grapeseed oil, sesame oil, and medium-chain triglyceride (MCT) oil), one or more antioxidants (e.g., tocopherols, ascorbyl palmitate, ascorbic acid and derivatives), one or more flavorings, and/or one or more pigments or colorants. The one or more exogenous oils can be present in an amount of about 0.01 to about 10% w/w.

The one or more antioxidants, one or more flavorings, one or more pigments can each independently be present in an amount of about 0.01 to about 5%.

In certain aspects, the composition comprises an antioxidant. Non-limiting examples of antioxidants are ascorbyl palmitate, butylated hydroxy anisole, butylated hydroxy toluene, propyl gallate, α-tocopherol, γ-tocopherol, mixed tocopherols, polyphenols and an ascorbate. In yet further aspects, the antioxidant is selected from the group consisting of a tocopherol, polyphenols and an ascorbate. The antioxidant can be present in amount from about 0.01 to about 10% w/w.

In some embodiments, the composition is substantially free of an exogenous oil.

In yet other aspects, the composition comprises an exogenous oil. The exogenous oil can be an MCT oil, an LCT oil, or a combination thereof. An oil comprising an MCT and/or LCT can be selected from the group consisting of borage oil, castor oil, coconut oil, cottonseed oil, soybean oil, safflower oil, sunflower oil, castor oil, corn oil, olive oil, palm oil, peanut oil, poppy seed oil, canola oil, hydrogenated soybean oil, hydrogenated vegetable oils, sesame oil, triolein, trilinolein, and trilinolenin. An oil comprising an MCT and/or LCT can also be selected from the group consisting of borage oil, castor oil, coconut oil, cottonseed oil, soybean oil, safflower oil, sunflower oil, castor oil, corn oil, olive oil, palm oil, almond oil, grapeseed oil, palm kernel oil, peanut oil, poppy seed oil, canola oil, hydrogenated soybean oil, hydrogenated vegetable oils, sesame oil, triolein, trilinolein, and trilinolenin, or any combination thereof. In certain specific embodiments, the exogenous oil is selected from the group consisting of almond oil, grapeseed oil, sesame oil, coconut oil, and palm kernel oil. The exogenous oil can be present in the composition in an amount from about 0.01 and about 10% w/w.

In certain specific aspects, the composition comprises Δ9-THC and the composition does not comprise an exogenous oil.

In additional embodiments, the composition comprises CBD and the composition comprises an exogenous oil.

The composition can further comprise one or more inactive ingredients, such as those selected from a group consisting of antiadherents, binders, coatings, disintegrants, flavors, colors, lubricants, glidants, sorbents, preservatives, sweeteners, edible carriers, and combinations thereof. Additional ingredients also include preservatives, absorption enhancers, coloring agents, pH modifiers, taste-masking agents, nutraceuticals, vitamins, supplements, and/or GRAS agents.

The composition can further comprise a pH adjusting agent. Exemplary pH adjusting agents are disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and combinations thereof. In some aspects, the pH adjusting agent is disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and combinations thereof.

Exemplary preservatives are methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzoate, calcium benzonate, sodium metabisulfite, propylene glycol, benzaldehyde, butylated hydroxytoluene, butylated hydroxyanisole, formaldehyde donors, essential oils, monoglyceride, and combinations thereof.

Exemplary sweeteners, flavoring and/or taste-masking agents include, for example, glucose, fructose, sucrose, sorbitol, sucralose, saccharin sodium, aspartame, neotame, acesulfame potassium, stevioside, sodium chloride, D-limonene, Luo Han Guo (monk) fruit extract powder, mannitol, xylitol, flavors fragrances and aroma, and combinations thereof. In one embodiment, the sweetener is selected from one or more of: acesulfame potassium, advantame, aspartame, neotame, saccharin, mannitol, Luo Han Guo (monk) fruit extract powder, sucralose, stevia, glucose, fructose, sucrose, sorbitol, or xylitol.

Exemplary nutraceuticals and supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. Dietary supplements and nutraceuticals are also disclosed in Physicians' Desk Reference for Nutritional Supplements, 1st Ed. (2001) and The Physicians' Desk Reference for Herbal Medicines, 1st Ed. (2001), both of which are also incorporated by reference. A nutraceutical or supplement, can also be referred to as phytochemicals or functional foods, is generally any one of a class of dietary supplements, vitamins, minerals, herbs, or healing foods that have medical or pharmaceutical effects on the body.

Exemplary nutraceuticals or supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., arginine, iso-leucine, leucine, lysine, methionine, phenylalanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and supplements also include bio-engineered foods genetically engineered to have a desired property, also known as “pharmafoods.”

In certain aspects, the composition is an oral dosage form, for example, the composition can be formulated as a tablet or a capsule. In yet other aspects, the composition is an oral dosage form, for example, the composition is formulated as a beverage additive powder.

The invention also includes methods for the preparation of the self-emulsifying, solid composition described herein. In certain aspects, the method is for the preparation of particles having a core-shell structure as described herein (wherein the surfactant and cannabinoid and/or terpene are present on the surface of the solid carrier) and the method comprises forming a solution by dissolving the cannabinoid and/or terpene in a solvent mixture to form a first solution (also referred to herein as “Solution A”). The solvent mixture comprises a solvent (optionally, a volatile solvent) and the surfactant. In this method, the solid carrier can be a water-soluble solid carrier or water-insoluble solid carrier and is insoluble in the first solution (Solution A). The method comprises:
i. mixing the solid carrier with the first solution to form a mixture, wherein the solid carrier is insoluble in the first solution;
ii. evaporating the solvent from the mixture; and
iii. collecting the particles.

As used herein, when a substance (for example, the solid carrier) is “insoluble” in a solvent or a solution, it is meant that the substance does not dissolve or does not fully dissolve in the solvent or solution, and/or the substance and the solvent or solution do not form a homogeneous phase at the molecular level, and/or that layer separation or phase separation occurs. The mixing step (step i) can, for example, be performed using a V-shaped mixer, a ploughshare mixer, or a granulator to coat the surface of the solid carrier. The particles or powder having the desired particle size fraction can be collected, for example, using an electric sieve (e.g., a 40 mesh electric sieve). In additional aspects, the solid composition that is prepared can comprise additional agents, additional components, additional inactive ingredients, by including those additional agents/components/inactive ingredients as a solute in the first solution. The additional solute can, for example, be an antioxidant, a flavoring, a sweetener, or a combination thereof. The first solution can also comprise an exogenous oil when it is desired that an exogenous oil be part of the solid composition; for example, when the cannabinoid comprises CBD. For preparation of particles having an amorphous matrix structure, a water-soluble solid carrier is used that is soluble in the first solution can be used. This method can comprise the steps of:
i. dissolving the cannabinoid or the terpene in a solvent mixture to form a first solution, wherein the solvent mixture comprises a solvent, optionally a volatile solvent, and the surfactant;
ii. mixing the water-soluble, solid carrier with the first solution to form a second solution, wherein the water-soluble, solid carrier is soluble in the first solution;
iii. drying the second solution on a surface or in a container to form an amorphous solid;
iv. breaking the amorphous solid into particles; and
v. collecting the particles.

The second solution is dried, for example, to form an amorphous film/cylinder/cake collapse using hot melt extrusion, spray drying, or lyophilization/freeze-drying. When the second solution is dried by hot melt extrusion then the solvent is water or another volatile solvent (for example, ethanol), or a mixture thereof, in which both the surfactant payload mix and solid carrier are soluble. When the second solution is dried by spray drying then then the solvent is water or another volatile solvent (for example, ethanol), or a mixture thereof (for example a mixture of water and ethanol) in which both the surfactant payload mix and solid carrier are soluble. When the second solution is dried by freeze-drying then the second solvent is water or another aqueous solvent. In these methods, the solvent is removed (e.g., by evaporation) and is not present in the formed solid composition.

The amorphous solid can be broken into particles using a mill, for example. As above, the particles or powder having the desired particle size fraction can be collected, for example, using an electric sieve (e.g., a 40 mesh electric sieve). In additional aspects, the solid composition that is prepared can comprise additional agents/components by including those additional agents/components as a solute in the first solution. The additional solute can, for example, be an antioxidant, a flavoring, a sweetener, or a combination thereof. The first solution can also comprise an exogenous oil when it is desired that an exogenous oil be part of the solid composition.

The invention also encompasses a method for the preparation of the amorphous solid without solvent, the method comprising:
a. mixing the cannabinoid or the terpene with the water-soluble solid carrier and forming an amorphous matrix by hot melt extrusion;
b. breaking the amorphous solid into particles; and
c. collecting the particles.

The invention also includes a self-emulsifying, solid composition prepared by a method described herein.

As described herein, the solid compositions described herein self-emulsify upon addition to an aqueous medium. Thus, the invention includes a method of dispersing or dissolving a cannabinoid or a terpene in an aqueous medium, the method comprising adding the self-emulsifying, solid composition described herein to the aqueous medium. The diameter of the self-emulsifying nanoparticles can be about 10 to about 800 nm, about 10 to about 500 nm, or about 10 to about 300 nm, and as further described herein. The solid composition dissolves rapidly upon addition the aqueous medium to form a solution. In certain aspects, the solid composition dissolves to form a solution within about two minutes, within about one minute, or within about 30 seconds of addition to an aqueous medium. In additional aspects, the composition comprising the cannabinoid dissolves within about two minutes, within about one minute, within about 30 seconds upon addition to the aqueous medium, wherein the solution comprises at least about 0.01 mg/ml cannabinoid.

The self-assembled nanoparticles can have a diameter of about 10 to about 800 nm, for example, as measured by Dynamic Light Scattering (DLS). In yet additional aspects, the solid formulation comprises Δ9-THC and the self-assembled nanoparticles have a diameter of about 10 to about 500 nm, or about 10 to about 200 nm, or about 10 to about 100 nm. In further aspects, the solid formulation comprises CBD and the self-assembled nanoparticles have a diameter of about 10 to about 500 nm, or about 20 to about 800 nm.

In certain specific aspects, the composition comprises Δ9-THC and the composition does not comprise an exogenous oil. In yet further embodiments, the composition comprises Δ9-THC, the composition does not comprise an exogenous oil, and addition of the composition to an aqueous medium results in the formation of self-assembled nanoparticles having a diameter of about 10 to about 20 nm as measured by DLS. As shown below, the diameter of the self-assembled nanoparticles formed by the composition comprising Δ9-THC in the absence of an exogenous is less than that formed by the same composition in the presence of an exogenous oil.

In additional embodiments, the composition comprises CBD and the composition further comprises an exogenous oil. In additional aspects, the composition comprises CBD, the composition comprises an exogenous oil, and addition of the composition to an aqueous medium results in the formation of self-assembled nanoparticles having a diameter of about 15 to about 150 nm as measured by DLS. As shown below, the diameter of self-assembled nanoparticles formed by the composition comprising CBD in the presence of an exogenous is less than that formed the same composition in the absence of an exogenous oil.

The aqueous medium to which the solid composition is added can be a beverage including, but not limited, drinking water. Beverages also include, for example, mineral water, coconut water, carbonated water, carbonated mineral water, tea, dairy milk, plant-based milk (such as almond milk, flax milk, cashew milk, and/or coconut milk), juices, beer (including non-alcoholic beer), sodas, and sports drinks). In some examples, when the aqueous suspension is added to an aqueous medium or a beverage, it emulsifies into a transparent or translucent emulsion. In yet additional aspects, the composition dissolves within about 30 seconds, about 25 seconds, or about 10 seconds upon addition to the aqueous medium. In one example of a beverage, the beverage is about 8 ounces (about 237 ml), e.g., 8 ounces of drinking water and the amount of cannabinoid and/or terpene dissolved in the beverage is an effective amount, e.g., at least about 0.5 mg, at least about 2 mg, at least about 5 mg, or at least about 10 mg. In certain aspects, the dissolution of the solid composition/formation of self-assembling micelles does not require agitation (e.g., shaking or stirring) for emulsification/dispersion.

In additional aspects, the aqueous medium is gastric fluid and/or intestinal fluid and optionally, the solid composition self-emulsifies after oral administration to a subject or patient. The solid composition can be administered to or by the subject for the treatment of a disease or condition, for alleviation of a symptom, or for inducing a psychoactive effect, for example. The invention thus encompasses a method of oral administration of a cannabinoid or a terpene to a subject, comprising orally administering a self-emulsifying solid composition described herein. The composition can be an oral dosage form such as a tablet or a capsule (including hard-shell capsules). Tablets include, for example, taste-masked tablets formulated with polymer coatings, effervescence and/or flavorings; and controlled or sustained release tablets, e.g., for targeted delivery to colon or intestine, including polymer coatings. Effervescents include, but are not limited to, sodium carbonate, potassium carbonate, magnesium carbonate, calcium carbonate, sodium bicarbonate, potassium bicarbonate, magnesium bicarbonate, and/or calcium bicarbonate. Non-limiting examples of polymer coatings including polymethacrylates such as those sold under the tradename Eudragit (see, e.g., Table 12 below). Tablets also include those for sublingual administration. The tablet can additionally be coated. Capsules including, for example, hard gelatin capsules negating the need for capsule sealing by use of solid, self-emulsifying formulations; and/or enteric-coated hard-shell capsules, e.g., for targeted delivery to colon or intestine. The composition for oral dosage form can also be a gummy. The solid composition can also, for example, be a beverage additive powder.

The amount of the solid composition in the oral dosage form (e.g., the tablet, powder, gummy, effervescent powder, etc.) can vary depending on the formulation. In certain aspects, the amount of the solid composition in the oral dosage form is between about 0.01 to about 99.9% by weight. In other examples, the solid composition is present in the oral dosage form in an amount of about 0.01 to about 20%, 10 to about 99%, 10 to about 80%, about 10 to about 60%, about 10 to about 50%, about 15 to about 45%, about 20 to about 60%, about 20 to about 40%, about 30 to about 60%, or about 35 to about 55% or about 40 to about 50% (wherein the percentages are by weight).

### Cannabinoids

The chemical structures of Δ9-THC, Δ8-THC, cannabidiol (CBD), cannabinol (CBN), cannabidivarin (CBDV), cannabigerol (CBG), tetrahydrocannabivarin (THCV), cannabicyclol (CBL), cannabichromene (CBC), cannabivarin (CBV), cannabigerovarin (CBGV), cannabichromevarin (CBCV), and cannabigerol monomethyl ether (CBGM), which can be used in the solid compositions described herein, are shown below:

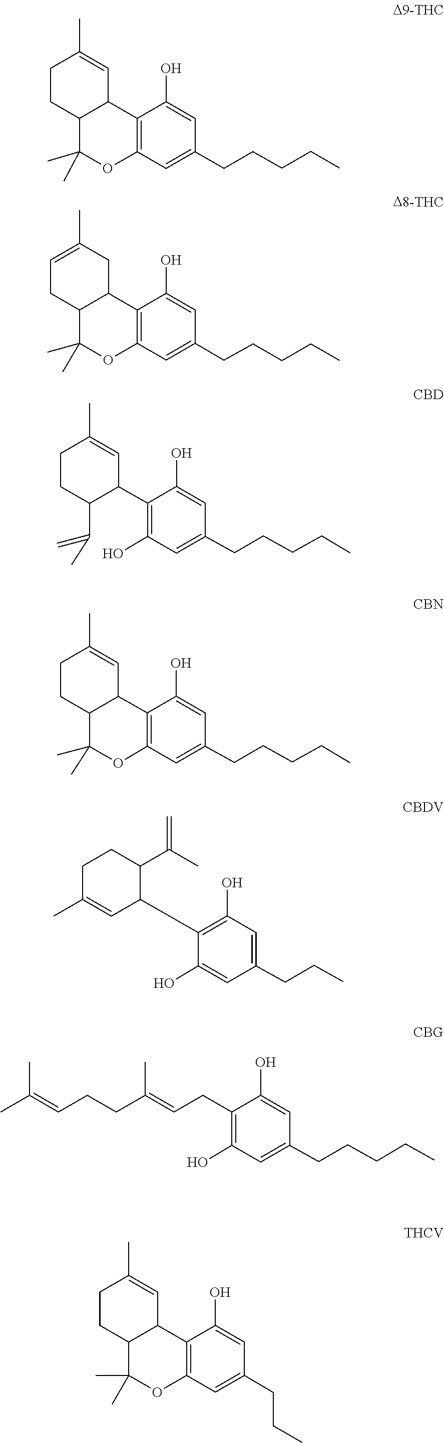

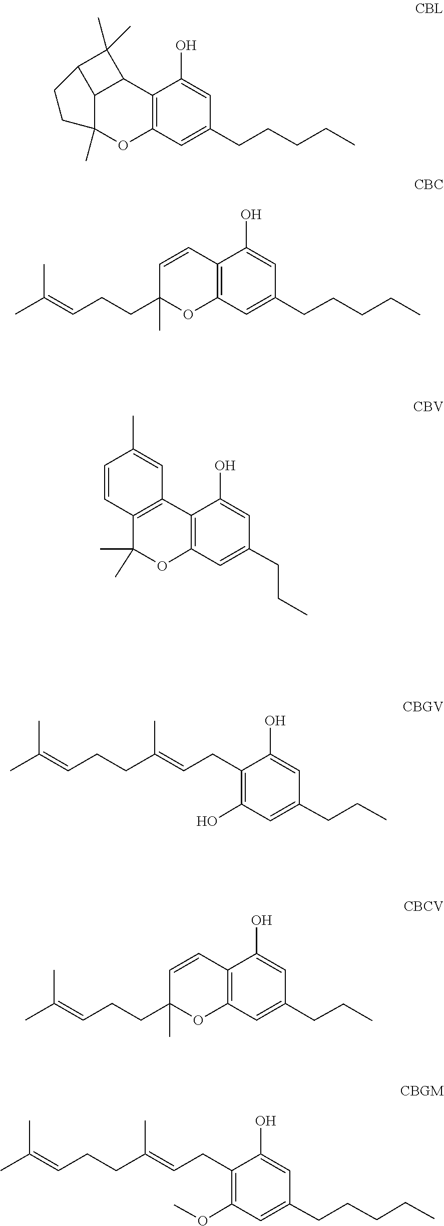

Certain cannabinoids, like Δ9-THC, can have three fused rings and these rings are referred to in the literature as the A-ring, B-ring and C-ring. For example, Formula (a) below shows the structure of THC, where the dashed line represents either a double bound between 8-9 (Δ8-THC) or between 9-10 (Δ9-THC). As illustrated below, certain cannabinoids lack one or two of rings A, B, or C, e.g., CBC (Formula (b)), CBL (Formula (c)) or CBD (Formula (d)).

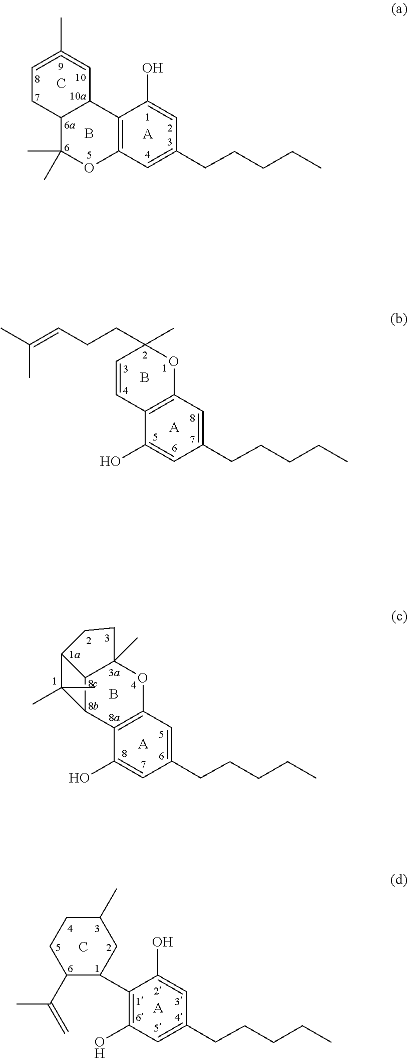

Cannabinoids that can be used in the methods and compositions of the present invention include, but are not limited, to: tetrahydrocannabinol (THC), Δ9-tetrahydrocannabinol (Δ9-THC), Δ8-tetrahydrocannabinol (Δ8-THC), tetrahydrocannabinolic acid (THCA), cannabinolic acid (CBNA), Δ8-tetrahydrocannabinol-dimethylheptyl, Δ9-tetrahydrocannabinol-dimethylheptyl, Δ9-tetrahydrocannabinol propyl analogue (THCV), 11-nor-9-carboxy-tetrahydrocannabinol, 5′-azido-Δ8-tetrahydrocannabinol, AMG-1, AMG-3, AM411, AM855, nabilone, HU-210, dexanabinol (HU-211), HU-308, O-1184, JWH-051, AM087, cannabivarin (CBV), tetrahydrocannabivarin (THCV), cannabichromevarin (CBCV), cannabigerol monomethyl ether (CBGM), cannabinol (CBN), cannabichromene (CBC), cannabichromene acid (CBCA) cannabichromene propyl analogue, cannabicyclol (CBL), levonantradol (CP 50556-1), 19,19-Dimethylheptyl-D-8-tetrahydrocannabinol-11-oic acid (CT-3), 9-carboxy-11-norcannabinol, 1′-oxocannabinol, 11-nor-Δ8-THC-9-carboxylic acid, 2′-carboxy-3′,4′,5′-trinor-Δ9-THC, 5′-carboxy-Δ9-THC, 9-carboxy-11-nor-Δ9-THC, 9-carboxy-11-nor-Δ8-THC, [(6aR,10aR)-3-[(1S,2R)-1,2-dimethylheptyl]-6a,7,10,10a-tetrahydro-6, 6,9-trimethyl-6H-dibenzo[b,d]pyran-1-ol], 9-carboxy-11-nor-(2 or 4)-chloro-Δ8-THC, 5′-Dimethylamino-Δ8-THC, 5′-methylamino-Δ8-THC, 5′-N-methyl-N-4-(7-nitrobenzofurazano)amino-Δ8-THC, (−)-trans-Δ8-THC, 5′-trimethylammonium-Δ8-THC phenolate, cannabidiolic acid (CBDA), Δ9-tetrahydrocannabiphorol (THCP), Δ9-tetrahydrocannabutol (THCB), cannabidiphorol (CBDP), cannabidibutol (CBDB), cannabigerolic acid (CBGA), 11-hydroxy-tetrahydrocannabinol, AM938, AM708, AM836, CP 55940, CP 55244, AM919, AM926, dimethylheptyl HHC, cannabidiol (CBD), cannabidivarin (CBDV), cannabigerovarin (CBGV), cannabidiol propyl analogue (CBDV), cannabigerol (CBG), cannabielsoin (CBE), cannabinodiol (CBDL), IP-751 (ajulemic acid), desacetyl-L-nantradol, CP 47497, cannabicyclohexanol (CP-47,497 C8 homolog), CP 47497 C6 homolog, CP 47497 C7 homolog, or CP 47497 C9 homolog, 10-hydroxycannabidiol, 1′-hydroxycannabinol, 11-hydroxycannabinol, 11-hydroxy-Δ9-THC, 1′-hydroxy-Δ9-THC (Isomer B), 11-hydroxy-Δ8-THC, 2′-hydroxy-Δ9-THC, 3′-hydroxy-Δ9-THC, 4′-hydroxy-Δ9-THC, 5′-hydroxy-Δ9-THC, 8α-hydroxy-Δ9-THC, 8β-hydroxy-Δ9-THC, 5′-Trimethylammonium-11-hydroxy-Δ8-THC phenolate, cannabinodiol (CBND), cannabitriol (CBTL), 8α-11-dihydroxy-Δ9-THC, or 8β-11-Dihydroxy-Δ9-THC.

Additional cannabinoids have also been described, for example, in Thakur et al. (2005). Structural Requirements for Cannabinoid Receptor Probes, HEP 168: 209-246; Seltzman et al. (1999). Structure and receptor activity for Classical Cannabinoids, Current Medicinal Chemistry 6: 685-704; Bow et al. (2016). The Structure-Function Relationships of Classical Cannabinoids: CB1/CB2 Modulation. Perspectives in Medicinal Chemistry 2016:8 17-39 doi: 10.4137/PMC. S32171; the contents of each of which are expressly incorporated by reference herein.

In a preferred embodiment, the cannabinoid is selected from the group consisting of THC, THCA, THCB, THCP, THCV, CBD, CBDA, CBDB, CBDP, CBDV, CBDL, CBC, CBCA, CBCV, CBCN, CBV, CBG, CBGA, CBGV, CBN, CBL, and CBE, or a combination of any of thereof. In certain additional aspects, the cannabinoid is selected from the group consisting of CBG, THCV, CBN, THC, CBC, CBD, and CBDV. In another embodiment, the cannabinoid is selected from the group consisting of THC, CBD, THCA, and CBDA, or a combination of any of thereof. In another embodiment, the cannabinoid is THC or CBD, or a combination thereof. In another embodiment, the THC is Δ9-THC or Δ8-THC, or a combination thereof. In another embodiment, the THC is Δ9-THC. In a further specific aspect, the cannabinoid is CBD. In yet an additional aspect, the formulation comprises a combination of Δ9-THC and CBD.

In additional aspects, the cannabinoid is a naturally occurring cannabinoid. Naturally occurring cannabinoids include cannabinoids that can be extracted from or isolated from the Cannabis sativa, Cannabis indica, or cannabis hybrid plants.

The compositions described herein can comprise a terpene in the absence of a cannabinoid, or in combination with a terpene.

Cannabinoids and terpenes can be purchased or synthesized using well-known techniques. Cannabinoids can be extracted from a plant using well-known methods. Specifically, cannabinoids and terpenes can be extracted from a plant of the Cannabis genus, e.g., Cannabis sativa, Cannabis indica, or Cannabis hybrid. Terpenes can also be extracted from a plant that is not a member of the Cannabis genus. Phytocannabinoids and terpenes may be extracted as terpene blends or, in the case of a cannabis species, as a cannabinoid or cannabinoid/terpene blend. The blends may be used directly or can be separated into individual or fewer components using distillation (e.g., short-path rotary distillation) or other techniques. The relative amount of each principal phytocannabinoid and/or terpene in the plant extract, e.g., cannabis extract, varies according to the cannabinoid and/or terpene profile and levels of the particular plants and methodology of extraction. Extracts comprising terpenes, e.g., extracts essentially free of cannabinoids, extracts that contain cannabinoids as a minor constituent, or extracts from a plant that is not a species of Cannabis (e.g., Cannabis sativa, Cannabis indica, Cannabis hybrid, or other), i.e., a non-Cannabis species, may be used individually or combined with one or more other active ingredients, e.g., cannabinoids or cannabinoid extracts.

Cannabinoids and/or terpenes can be obtained by separating resins from leaves or leaves and flowers of cannabis plants by solvent extraction. Extracts derived from cannabis plants include primary extracts prepared by such processes as, for example, maceration, percolation, and solvent extraction. Solvent extraction can be carried out using a solvent that dissolves cannabinoids/cannabinoid acids, such as for example C1 to C5 alcohols (e.g. ethanol, methanol), C3-C12 alkanes (e.g. hexane, butane or propane), Norflurane (HFA134a), HFA227, and carbon dioxide. General protocols for the preparation of extracts of cannabis plant material are described in U.S. Pat. App. Pub. No 20060167283 (WO 02/064109), which is incorporated herein by reference. Carbon dioxide provides another method to extract cannabinoid/terpene resins from cannabis plant material. Sub Critical (Liquid) or Supercritical CO₂ is forced through the plant matter, which separates the cannabinoid/terpenes from the plant matter resulting in a transparent, amber oil. Primary extracts obtained by supercritical fluid extraction (SFE) may undergo an ethanolic precipitation step in order to remove less polar, plant derived impurities (e.g., lipids). The extracts obtained by supercritical fluid extraction (SFE) may undergo a secondary extraction, e.g. an ethanolic precipitation, to remove non-cannabinoid/terpene materials. In a preferred embodiment, light petroleum gas extraction, using a LHBES (light hydrocarbon butane extraction system) 1300/C from ExtractionTek Solutions is used to extract cannabinoids from cannabis plant material.

A modified extraction process consists of decarboxylating the starting concentrate at 300° F. until fully converted and the bubbling stops. Once the oil is decarboxylated, it is run through the VTA-VKL 70-5 short path rotary distillation plant twice. The first run separates the heavy terpenes and lighter terpenes from the cannabinoids and waste material. The cannabinoids and waste are run through again with a higher vacuum and higher temperature to separate the cannabinoids from the remaining waste. The waste is collected and run again in a larger batch to extract all cannabinoids and terpenes. The VTA-VKL 70-5 short path rotary distillation plant uses a top stirring rotary column to wipe incoming product into a thin film for better heat distribution and evaporation. The inner condensing column is set to condense the cannabinoids into liquids. The waste and cannabinoids are diverted into the two dispensing arms for collection into receiving vessels. The light terpenes are collected in a receiving flask attached to the inline chiller on the plant. The system (except for feed vessel) are under vacuum during the operation. The vacuum for the first run should be between 0.5-0.7 mbar. For the second run, pressure should be between 0.5-0.07 mbar.

In one embodiment, the cannabinoid is in the form of an extract from a cannabis plant comprising a cannabinoid, i.e., a “cannabinoid extract”. The composition can comprise a terpene in the form of an extract, in the presence or absence of a cannabinoid. In some embodiments, the terpene is in the form of an extract from cannabis or other plant comprising a terpene, i.e., a “terpene extract” In a further embodiment, the cannabinoid or terpene extract is from a cannabis plant selected from Cannabis sativa, Cannabis indica, or Cannabis hybrid. In one embodiment, the cannabinoid or terpene extract is an extract of Cannabis sativa. In another embodiment, the cannabinoid or terpene extract is an extract of Cannabis indica. In another embodiment, the cannabinoid or terpene extract is an extract of Cannabis hybrid. In another embodiment, the cannabinoid or terpene extract is a distillate. In a further embodiment, the cannabinoid distillate is the product of short path distillation of a cannabinoid extract. In a further embodiment, the cannabinoid or terpene is synthetic.

In further embodiments, the cannabinoid extract comprises total cannabinoid(s) in an amount selected from: 50-75 wt %, 50-99 wt %, 75-99 wt %, 75-95 wt %, 80-99 wt %, 85-99 wt %, 90-99 wt %, 85-95 wt %, 90-95 wt %, or >99 wt % total cannabinoid(s). In further embodiments, the total concentration of cannabinoid(s) in a composition of the present invention is 1-200 mg/mL. In further embodiments, the total concentration of cannabinoid(s) in a composition of the present invention is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In another embodiment, the total concentration of cannabinoid(s) in a composition of the present invention is <0.001 mg/mL, 0.001-0.01 mg/mL, or 0.01-1 mg/mL.

In one embodiment, the composition described herein comprises at least one terpene. In one embodiment, the terpene is found in Cannabis sativa, Cannabis indica, or Cannabis hybrid. In a further embodiment, the terpene is extracted from a plant species, preferably a species of Cannabis (e.g., Cannabis sativa, Cannabis indica, Cannabis hybrid or other). In a further embodiment, the terpene is synthetic. In a further embodiment, the terpene is selected from any, one, two, three, four, five, six, seven, eight, nine, ten, eleven, twelve, thirteen, fourteen, fifteen, sixteen, seventeen, eighteen, nineteen, twenty, or more of the group consisting of: alpha-bisabolol, alpha-phellandrene, alpha-pinene, alpha-terpinene, alpha-terpineol, beta-caryophyllene, α-pinene, beta-pinene, borneol, cadinene, camphene, camphor, carvacrol, β-caryophyllene, caryophyllene acetate, caryophyllene oxide, cedrane, citral, citronellol, dextro carvone, dextro fenchone, eucalyptol (1,8-cineole), eugenol, farnesene, gama-3-carene, gamma-terpinene, geraniol, geranyl acetate, guaiene, humulene, isopulegol, limonene, linalool, linalyl acetate, menthol, myrcene, β-myrcene, nerol, nerolidol, ocimene, ocimene, p-cymene, phytol, pulegone, terpineol, terpinen-4-ol, terpinolele, terpinolene, thymol, valencene, valencene,1-menthol, and combinations thereof.

As described above, the compositions described herein can comprise a terpene alone or in combination one or more cannabinoid. In a further embodiment, the at least one terpene is any one, two, three, four, five, six, or all six terpenes selected from the group consisting of beta-caryophyllene, linalool, limonene, alpha-pinene, eucalyptol, and myrcene. In a further embodiment, the at least one terpene is any one, two, three, four, or all five selected from beta-caryophyllene, linalool, limonene, alpha-pinene, or eucalyptol. In yet an additional aspect, the at least one terpene is any one, two, three, four, or all five selected from beta-caryophyllene, α-pinene, β-pinene, eucalyptol, and limonene. In certain embodiments, the at least one terpene is a blend comprising beta-caryophyllene, α-pinene, β-pinene, eucalyptol, and limonene. In yet further aspects, the blend comprises about 40% w/w beta-caryophyllene, about 15% w/w α-pinene, about 15% w/w β-pinene, about 10% w/w eucalyptol, and about 20% w/w limonene.

In some embodiments, the cannabinoid is Δ9-THC and the dose of the Δ9-THC administered to the subject is effective to induce a psychoactive effect. In additional aspects, the cannabinoid is Δ9-THC, and the amount of the cannabinoid is an effective amount or a therapeutically effective amount. In yet further aspects, the cannabinoid is CBD and the amount of the CBD is an effective amount or a therapeutically effective amount. In yet additional aspect, the formulation comprises a Δ9-THC and CBD, wherein the Δ9-THC and CBD are each present in an effective amount or a therapeutically effective amount.

The dose of cannabinoid is related to the concentration of the cannabinoid in the formulation (for example, % w/w). In certain aspects, the dose of the cannabinoid administered is greater than about 0.2 mg, greater than about 0.35 mg, greater than about 0.5 mg.

In one embodiment, the composition, e.g., cannabinoid composition, of the present invention has an onset of action within 15 minutes, 15-20 minutes, 20 minutes, 25 minutes, 30 minutes, or within 45 minutes post administration.

In one embodiment, the composition, e.g., cannabinoid composition, of the present invention has a peak time within 90 minutes, within 80 minutes, within 70 minutes, within 60-70 minutes, within 60 minutes, within 50 minutes, within 45-60 minutes, within 45 minutes, within 40 minutes, or within 30 minutes post administration.

### Additional Actives

In one embodiment, the composition described herein comprises at least two active ingredients, wherein at least one of the active ingredients is the cannabinoid or a terpene. In addition to the cannabinoid, the composition may contain, e.g., one or more additional cannabinoids, terpenes, or other additional non-cannabinoid active ingredient(s). In one embodiment, at least one of the other additional active ingredients, i.e., in addition to the cannabinoid, is selected from one or more cannabinoid, cannabinoid extract, terpene, terpene extract, anti-insomnia, anti-tussive, opioid analgesic, decongestant, non-opioid analgesic/anti-inflammatory drug, anti-migraine drug, anti-emetic, anti-histamine, proton pump inhibitor, H₂ antagonist/H₂ blocker, tranquilizer, anticonvulsant, hypnotic, muscle relaxant, anti-psychotic, anti-diarrheal, Attention Deficit and Hyperactivity Disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, or a combination thereof.

In one embodiment, the composition described herein comprises at least two active ingredients, wherein at least one of the active ingredients is a terpene. In addition to the terpene, the composition may contain, e.g., one or more additional cannabinoids, terpenes, or other additional non-cannabinoid active ingredient(s). In one embodiment, at least one of the other additional active ingredients, i.e., in addition to the terpene, is selected from one or more cannabinoid, cannabinoid extract, terpene, terpene extract, anti-insomnia, anti-tussive, opioid analgesic, decongestant, non-opioid analgesic/anti-inflammatory drug, anti-migraine drug, anti-emetic, anti-histamine, proton pump inhibitor, H₂ antagonist/H₂ blocker, tranquilizer, anticonvulsant, hypnotic, muscle relaxant, anti-psychotic, anti-diarrheal, Attention Deficit and Hyperactivity Disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, or a combination thereof.

In another embodiment, the additional active ingredient(s) comprises an anti-insomnia drug. In further embodiments, the anti-insomnia drug is selected from any one or more of: melatonin, trazodone, zolpidem, temazepam, elprazolam, amitriptyline, halcion, lorazepam, clonazepam, Intermezzo, eszopiclone, diphenhydramine, doxepin, mirtazapine, gabapentin, doxylamine, quetiapine, flurazepam, estazolam, olanzapine, Seconal, triazolam, zaleplon, secobarbital, chloral hydrate, oxazepam, quazepam, ramelteon, suvorexant, butabarbital, pentobarbital, phenobarbital, phenyltoloxamine, amobarbital, diphenhydramine, dimenhydrinate, diphenhydramine/magnesium salicylate, diphenhydramine/naproxen, diphenhydramine/asprin, diphenhydramine/paracetamol, diphenhydramine/ibuprofen, or tasimelteon.

In some embodiments, the additional active ingredient(s) comprise an anti-tussive. In further embodiments, the anti-tussive is selected from any one or more of: benzonatate, caramiphen edisylate, chlophedianol, codeine, dextromethorphan hydrobromide, hydrocodone, levopropoxyphene, morphine, codeine, ethylmorphine, dihydrocodeine, benzylmorphine, laudanum, dihydroisocodeine, nicocodeine, nicodicodeine, hydrocodone, hydromorphone, acetyldihydrocodeine, thebacon, diamorphine (heroin), acetylmorphone, noscapine, or pholcodine.

In additional embodiments, the additional active ingredient(s) comprise an opioid analgesic. In further embodiments, the opioid analgesic is selected from any one or more of: alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, or tramadol.

In additional embodiments, the additional active ingredient(s) comprise a decongestant. In further embodiments, the decongestant is selected from any one or more of: pseudoephedrine hydrochloride, phenylephrine bitartrate, phenylephrine hydrochloride or pseudoephedrine sulfate.

In certain additional embodiments, the additional active ingredient(s) comprise a non-opioid analgesic/anti-inflammatory drug. In further embodiments, the non-opioid analgesic/anti-inflammatory is selected from any one or more of: acetaminophen or a non-steroidal anti-inflammatory agent selected from the group consisting of aspirin, celecoxib, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, or isoxicam.

In additional aspects, the additional active ingredient(s) comprise an anti-migraine drug. In further embodiments, the anti-migraine drug is selected from any one or more of: 2-bromo-LSD, acetaminophen/dichloralphenazone/isometheptene mucate, almotriptin, alniditan, amidrine, avitriptan, caffeine/ergotamine, calcitonin gene-related peptide receptor antagonist, clonidine, dasolampanel, dihydroergotamine, dimetotiazine, donitriptan, dotarizine, eletriptan, ergotamine, ergotamine/chlorcyclizine/caffeine, flumedroxone acetate, iprazochrome, lasmiditan, lisuride, lomerizine, methysergide, migraleve, naratriptan, naproxen, naproxen/sumatripta, olcegepant, oxetorone, paracetamol/metoclopramide, prochlorperazine, promethazine, proxibarbital, rimegepant, rizatriptan, selurampanel, sumatriptan, telcagepant, tezampanel, topiramate, or zolmitriptan.

In one embodiment, the additional active ingredient(s) comprise an anti-emetic. In further embodiments, the anti-emetic is selected from any one or more of: dolasetron, granisetron, ondansetron, tropisetron, palonosetron, mirtazapine, metoclopramide, cyclizine, diphenhydramine, dimenhydrinate, meclizine, promethazine, or hydroxyzine.

In additional embodiments, the additional active ingredient(s) comprise an anti-histamine. In further embodiments, the anti-histamine is selected from any one or more of: diphenhydramine, loratadine, desloratadine, meclizine, fexofenadine, pheniramine, cetirizine, promethazine, brompheniramine, clemastine fumarate or chlorpheniramine.

In some embodiments, the additional active ingredient(s) comprise a proton pump inhibitors (PPI). In further embodiments, the PPI is selected from any one or more of: omeprazole, esomeprazole, pantoprazole, lansoprazole, or rabeprazole.

In some embodiments, the additional active ingredient(s) comprise a H₂ antagonist/H₂ blocker. In further embodiments, the H₂ antagonist/H₂ blocker is selected from any one or more of: cimetidine, ranitidine, or famotidine.

In some embodiments, the additional active ingredient(s) comprise a tranquilizer. In further embodiments, the tranquilizer is selected from any one or more of: amobarbital, pentobarbital, secobarbital, phenobarbital, clonazepam, diazepam, estazolam, flunitrazepam, lorazepam, midazolam, nitrazepam, oxazepam, triazolam, temazepam, chlordiazepoxide, or alprazolam.

In some embodiments, the additional active ingredient(s) comprise an anticonvulsant. In further embodiments, the anti-convulsant is selected from any one or more of: elbamate, carbamazepine, oxcarbazepine, vigabatrin, progabide, tiagabine, topiramate, gabapentin, pregabalin, ethotoin, phenytoin, valproic acid, or lamotrigine.

In some embodiments, the additional active ingredient(s) comprise a hypnotic. In further embodiments, the hypnotic is selected from any one or more of: zolpidem, zaleplon, zopiclone, or eszopiclone.

In some embodiments, the additional active ingredient(s) comprise a muscle relaxant. In further embodiments, the muscle relaxant is selected from any one or more of: methocarbamol, carisoprodol, chlorzoxazone, cyclobenzaprine, gabapentin, metaxalone, or orphenadrine.

In some embodiments, the additional active ingredient(s) comprise an anti-psychotic. In further embodiments, the anti-psychotic is selected from any one or more of: haloperidol, droperidol, chlorpromazine, fluphenazine, perphenazine, prochlorperazine, thioridazine, trifluoperazine, mesoridazine, promazine, triflupromazine, levomepromazine, methotrimeprazine, pimozide, chlorprothixene, flupenthixol, thiothixene, zuclopenthixol, clozapine, olanzapine, risperidone, quetiapine, ziprasidone, amisulpride, asenapine, or paliperidone.

In some embodiments, the additional active ingredient(s) comprise an anti-diarrheal. In further embodiments, the anti-diarrheal is bismuth subsalicylate or loperamide.

In some embodiments, the additional active ingredient(s) comprise an Attention Deficit and Hyperactivity Disorder (ADHD) drug. In further embodiments, the ADHD drug is selected from any one or more of: methylphenidate, dextroamphetamine sulfate, amphetamine, or atomoxetine hydrochloride.

In some embodiments, the additional active ingredient(s) comprise an anti-Parkinsons disease drug. In further embodiments, the anti-Parkinson disease drug is selected from any one or more of: amantadine, Apokyn, apomorphine, bromocriptine, carbidopa/levodopa, Cycloset, Duopa, entacapone/levodopa/carbidopa, Gocovri, levodopa, Mirapex, Mirapex ER, Neupro, Parlodel, pramipexole, Requip, Requip XL, ropinirole, rotigotine, Rytary, Sinemet, Sinemet CR, or Stalevo.

In some embodiments, the additional active ingredient(s) comprise a benzodiazepine. In further embodiments, the benzodiazepine is selected from any one or more of: alprazolam, bromazepam, chlordiazepoxide, clorazepate, diazepam, estazolam, flurazepam, halazepam, ketazolam, lorazepam, nitrazepam, oxazepam, prazepam, quazepam, temazepam, and triazolam.

In some embodiments, the additional active ingredient(s) comprise a benzodiazepine antagonist. In further embodiments, the benzodiazepine antagonist is flumazenil.

In some embodiments, the additional active ingredient(s) comprise a barbiturate. In further embodiments, the barbiturate is selected from any one or more of: amobarbital, aprobarbotal, butabarbital, butalbital, methohexital, mephobarbital, metharbital, pentobarbital, phenobarbital, and secobarbital.

In some embodiments, the additional active ingredient(s) comprise a barbiturate antagonist. In further embodiments, the barbiturate antagonist is an amphetamine.

In some embodiments, the additional active ingredient(s) comprise a stimulant. In further embodiments, the stimulant is selected from caffeine or an amphetamine, such as amphetamine, dextroamphetamine resin complex, dextroamphetamine, methamphetamine, or methylphenidate.

In some embodiments, the additional active ingredient(s) comprise a stimulant antagonist. In further embodiments, the stimulant antagonist is a benzodiazepine.

In one embodiment, the additional active ingredient(s) comprise an antidepressant. In further embodiments, the antidepressant is selected from any one or more of: agomelatine, Allegron (see nortriptyline), amitriptyline, Anafranil (see clomipramine), Brintellix (see vortioxetine), Cipralex (see escitalopram), Cipramil (see citalopram), citalopram, clomipramine, Cymbalta (see duloxetine), Depefex XL (see venlafaxine), dosulepin, doxepin, duloxetine, Edronax (see reboxetine), Efexor XL (see venlafaxine), escitalopram, Faverin (see fluvoxamine), fluoxetine, fluvoxamine, Foraven XL (see venlafaxine), imipramine, isocarboxazid, lofepramine, Lomont (see lofepramine), Lustral (see sertraline), Manerix (see moclobemide), mianserin, mirtazapine, moclobemide, Molipaxin (see trazodone), Nardil (see phenelzine), nortriptyline, Oxactin (see fluoxetine), Parnate (see tranylcypromine), paroxetine, phenelzine, Politid XL (see venlafaxine), Prothiaden (see dosulepin), Prozac (see fluoxetine), Prozep (see fluoxetine), reboxetine, Seroxat (see paroxetine), sertraline, Sinepin (see doxepin), Sunveniz XL (see venlafaxine), Surmontil (see trimipramine), Tofranil (see imipramine), Tonpular XL (see venlafaxine), tranylcypromine, trazodone, trimipramine, Triptafen, Valdoxan (see agomelatine), Venadex XL (see venlafaxine), Venaxx XL (see venlafaxine), venlafaxine, Venlalic XL (see venlafaxine), ViePax (see venlafaxine), vortioxetine, Zispin (see mirtazapine). In preferred embodiments, the antidepressant is selected from any one or more of: citalopram (Celexa), escitalopram (Lexapro), fluoxetine (Prozac), fluvoxamine (Luvox), paroxetine (Paxil), sertraline (Zoloft), desvenlafaxine (Pristiq), duloxetine (Cymbalta), levomilnacipran (Fetzima), milnacipran (Ixel, Savella), venlafaxine (Effexor), reboxetine (Edronax), teniloxazine (Lucelan, Metatone), or viloxazine (Vivalan).

In one embodiment, the additional active ingredient(s) comprise a nutraceutical. In further embodiments, the nutraceutical is selected from any one or more of: 5-methyltetrahydrofolic acid, ademetionine, adenine, adenosine monophosphate, alfacalcidol, alpha-linolenic acid, ATP, beta carotene, biotin, calcidiol, calcitriol, castor oil, cholecalciferol, choline, chondroitin sulfate, coenzyme A, coenzyme Q10, resveratrol, creatine, curcumin, cyanocobalamin, cystine, dihomo-gamma-linolenic acid, ephedra, ergocalciferol, eucalyptol, fish oil, folic acid, ginkgo biloba, ginkgolide-A, ginkgolide-B, ginkgolide-C, ginkgolide-J, ginkgolide-M, ginseng, ginsenoside C, ginsenoside Rb1, ginsenoside Rg1, glutamic acid, glutathione, glycine, glycine betaine, histidine, hyperforin, icosapent, icosapent ethyl, inulin, kava, krill oil, L-Alanine, L-Arginine, L-Asparagine, L-Aspartic Acid, L-Citrulline, L-Cysteine, L-Glutamine, L-Isoleucine, L-Leucine, L-Lysine, L-Phenylalanine, L-Proline, L-Threonine, L-Tryptophan, L-Tyrosine, L-Valine, lipoic acid, lutein, melatonin, menadione, methionine, N-Acetylglucosamine, NADH, niacin, octacosanol, omega-3 fatty acids, omega-6 fatty acids, ornithine, oxitriptan, oxogluric acid, pantothenic acid, phosphatidyl serine, phosphocreatine, prasterone, pyridoxal, pyridoxal phosphate, pyridoxine, pyruvic acid, riboflavin, sage oil, serine, serotonin, sesame oil, sinecatechins, spermine, St. John's Wort, succinic acid, taurine, tetrahydrofolic acid, thiamine, tretinoin, tyramine, ubidecarenone, ubiquinol, vitamin A, vitamin C, vitamin D, vitamin E, or vitamin K.

In additional embodiments, the additional active ingredient(s) comprise nicotine. In another embodiment, the additional active ingredient(s) comprise a BCS Class II active ingredient. In further embodiments, the BCS Class II active ingredient is selected from any one or more of following: aceclofenac, albendazole, amiodarone, atorvastatin, azithromycin, bicalutamide, bisacodyl, cabergoline, candesartancilexetil, carbamazepine, carvedilol, cefditoren, celecoxib, chloroquine, chlorpromazine, cilostazol, ciprofloxacin, cisapride, clarithromycin, clofazimine, clopidogrel, clozapine, cyclosporine, cyproterone, danazol, dapsone, diazepam, diclofenac, diflunisal, digoxin, diloxanide, ebastine, efavirenz, epalrestat, eprosartan, erythromycin, ethylicosapentate, ezetimibe, fenofibrate, flurbiprofen, furosemide, gefitinib, gliclazide, glimepiride, glipizide, glyburide, glyburide(glibenclamide), griseofulvin, haloperidol, hydroxyzine, ibuprofen, imatinib, indinavir, indomethacin, irbesartan, isotretinoin, itraconazole, ketoconazole, ketoprofen, lamotrigine, lansoprazolei, lopinavir, loratadine, lorazepam, lovastatin, mebendazole, medroxyprogesterone, meloxicam, menatetrenone, metaxalone, metoclopramide, mosapride, mycophenolatemofetil, nabumetone, naproxen, nelfinavir, nevirapine, nicergoline, niclosamide, nifedipine, nimesulide, ofloxacin, olanzapine, orlistat, oxaprozin, phenazopyridine, phenytoin, pioglitazone, piroxicam, pranlukast, praziquantel, pyrantel, pyrimethamine, quetiapine, quinine, raloxifene, rebamipide, retinol, rifampicin, risperidone, ritonavir, rofecoxib, saquinavir, simvastatin, sirolimus, spironolactone, sulfasalazine, tacrolimus, talinolol, tamoxifen, telmisartan, teprenone, terfenadine, ticlopidine, tocopherolnicotinate, tosufloxacin, triflusal, ursodeoxycholicacid, valproicacid, valsartan, verapamil, warfarin, or zaltoprofen.

In another embodiment, at least one additional active ingredient is a BCS Class IV active ingredient. In further embodiments, the BCS Class IV active ingredient is selected from any one or more of following: acetaminophen (paracetamol), acetazolamide, acetylsalicylic acid, acyclovir, allopurinol, aluminium hydroxide, amoxicillin, azathioprine, cefdinir, cefixime, cefotiam, cefpodoxime, cefuroxime axetil, dapsone, dexamethasone, doxycycline, famotidine, folic acid, hydrochlorothiazide, l-carbocysteine, levodopa, linezolid, mesalamine, methylphenidate, metronidazole, modafinil, nalidixic acid, nitrofurantoin, nystatin, oxcarbazepine, oxycodone, phenobarbital, propylthiouracil, roxithromycin, sulfadiazine, sulfamethoxazole, sulpiride, sultamicillin, theophylline, or trimethoprim.

In one embodiment, the combined active ingredients in a composition of the present invention have synergistic activity, as compared to the additive activity of equivalent compositions comprising each active ingredient alone.

### Additional Optional Additives/Ingredients

In some aspects, the composition can comprise at least one fatty acid, at least one monoglyceride, at least one diglyceride, or at least one triglyceride, or a combination thereof. In one embodiment, the fatty acid, monoglyceride, diglyceride, triglyceride, or a combination thereof is an oil. In a further embodiment, the oil is selected from anise oil, apricot kernel oil PEG-6 esters, apricot kernel oil, beeswax, borage oil, canola oil, castor oil, polyoxyl 35 castor oil, polyoxyl 40 hydrogenated castor oil, polyoxyl 40 castor oil, polyoxyl 60 hydrogenated castor oil, hydrogenated castor oil, polyoxyl 60 castor oil, cinnamon oil, clove oil, coconut oil fractioned, coconut oil, coconut oil-lecithin, coriander oil, corn oil PEG-6 esters, corn oil PEG-8 esters, corn oil, cottonseed oil hydrogenated, cottonseed oil, cottonseed oil, hydrogenated soybean oil, hydrogenated vegetable oils, kernel oil PEG-6 esters, kernel oil, lemon oil, mineral oil (light), mineral oil, neutral oil, nutmeg oil, olive oil PEG-6 esters, olive oil, orange oil, palm kernel oil PEG-6 esters, palm kernel oil, palm kernel oil/palm kernel oil hydrogenated, palm fruit oil, peanut oil PEG-6 esters, peanut oil, peppermint oil, poppy seed oil, safflower oil, soybean oil hydrogenated, soybean oil refined, soybean oil, sunflower oil, triisostearin PEG-6 esters, vegetable oil hydrogenated, vegetable oil PEG esters, vegetable oil, vegetable oils glyceride hydrogenated, or a mixture thereof.

In one embodiment, the fatty acid, monoglyceride, diglyceride, triglyceride, or a combination thereof is a fat. In another embodiment, the fatty acid, monoglyceride, diglyceride, triglyceride, or a combination thereof is exogenously added fatty acid, monoglyceride, diglyceride, triglyceride, or a combination thereof. The term “exogenously added,” as used herein, means other than any fatty acids, monoglycerides, diglycerides, triglycerides, or combinations thereof, that were originally present in a cannabis plant, or other plant extract, and remains in the extract, e.g., a cannabinoid extract, after the extraction/distillation process. For clarity, pressed cannabis/hemp seed oil added to a composition of the present invention is exogenously added. In one embodiment, the only exogenously added fatty acid, monoglyceride, diglyceride, triglyceride, or a combination thereof, is a flavoring oil, e.g., flavor compounds diluted with and MCT or other oil. In a further embodiment, the flavoring oil is an essential oil. In a further embodiment, the essential oil is produced by distillation (e.g., steam distillation), solvent extraction (example, a hydrocarbon such as hexane or supercritical carbon dioxide), or by expression.

In another embodiment, the monoglyceride, diglyceride, or triglyceride is a medium chain monoglyceride, diglyceride, or triglyceride and/or a long chain monoglyceride, diglyceride triglyceride. In a further embodiment, the triglyceride is a medium chain triglyceride (MCT). In another further embodiment, the triglyceride is a long chain triglyceride (LCT).

The medium chain triglycerides (MCT) of the present invention are triglycerides whose fatty acids have an aliphatic tail of 6-12 carbon atoms. The MCT may be a single MCT or a mix of MCT. In one embodiment, the MCT is formed from fatty acids having from C6 to C8, C8 to C10, C10 to C12, or C8 to C12 carbon atoms. The fatty acids of the MCT may be saturated, mono-unsaturated, and/or poly-unsaturated fatty acids. In one embodiment 80 to 100% of the medium chain fatty acids are saturated, 0 to 10% are monounsaturated, and 0 to 5% are polyunsaturated. Preferred medium chain fatty acids include caproic acid, caprylic acid, capric acid, and mixtures thereof. An oil comprising MCT, may comprise at least 5 wt % medium chain triglycerides, e.g., coconut oil, or palm kernel oil. In one embodiment, the oil comprising an MCT is coconut oil. MCT may be in the form of oil that is enriched or fractionated to increase the concentration of medium chain triglycerides. In one embodiment, the MCT is fractionated coconut oil (e.g., glyceryl tricaprylate or NATURE′S OIL MCT). Medium chain triglycerides may also be formed by esterifying glycerol with mixtures of C6-C12 fatty acids, e.g., C8-C10 fatty acids such as caprylic (C:8) and capric (C:10) fatty acids fractionated from coconut or palm kernel oils.

The long chain triglycerides (LCT) of the present invention are triglycerides whose fatty acids have an aliphatic tail of 13-24 carbon atoms. The LCT may be a single LCT or a mix of MCT. In one embodiment, the LCT is formed from long chain fatty having from C14 to C16, C16 to C18, C18 to C20, C14 to C20, or C20 to C24 carbon atoms. The fatty acids of the LCT may be saturated, mono-unsaturated, and poly-unsaturated fatty acids. In one embodiment 5 to 25% of the long chain fatty acids are saturated, 15 to 80% are monounsaturated, and 15 to 80% are polyunsaturated. The oil comprising an LCT may comprise at least 5 wt % long chain triglycerides, e.g., olive oil, poppy seed, safflower, sunflower, corn, and soybean oils, sesame oil, or castor oil. LCT may be in the form of oil that is enriched or fractionated to increase the concentration of long chain triglycerides. In one embodiment, the LCT is olive oil.

The oil comprising an MCT and/or LCT may be selected from the group consisting of borage oil, castor oil, coconut oil, cottonseed oil, soybean oil, safflower oil, sunflower oil, castor oil, corn oil, olive oil, palm oil, peanut oil, poppy seed oil, canola oil, hydrogenated soybean oil, hydrogenated vegetable oils, sesame oil, triolein, trilinolein, and trilinolenin.

As described above, the compositions may include one or more antioxidant. Antioxidants include ascorbic acid, ascorbyl palmitate, butylated hydroxy anisole, butylated hydroxy toluene, propyl gallate, α-tocopherol, α-tocopherol, and mixed tocopherols.

A composition may comprise chelating agents in a final range of about 0.01% to about 0.5% w/v. Examples of chelating agents include ethylenediaminetetraacetic acid (EDTA), phosphoric acid, polyphosphates, polysaccharides, citric acid and combinations thereof.

A composition may also additionally comprise inactive ingredients selected from a group consisting of co-solvents, dispersing agents, emulsifiers, flavors or flavorants (including, but not limited to, potassium, advantame, aspartame, neotame, saccharin, mannitol, Luo Han Guo (monk) fruit extract powder, sucralose, stevia, sodium chloride, glucose, fructose, sucrose, sorbitol, orxylitol, maple syrup, glucose syrup, tapioca syrup, corn syrup, high fructose corn syrup, golden syrup, cane syrup, agave syrup, as well as Food/pharmaceutical grade, natural/artificial flavor, bitterness modifier (FONA) powder; or a combination of any of thereof), humectants, lubricants (including, but not limited to, polyethylene glycol, magnesium stearate, and/or sodium stearyl fumarate; or a combination of any of thereof), preservatives, sorbents, suspension aids, sweeteners (potassium, advantame, aspartame, neotame, saccharin, mannitol, Luo Han Guo (monk) fruit extract powder, sucralose, stevia, glucose, fructose, sucrose, sorbitol, or xylitol; or a combination of any of thereof), tonicity modifiers, acidifiers (including, but not limited to, citric acid, malic acid, tartaric acid, fumaric acid, adipic acid, boric acid; or a combination of any of thereof), defoamers (including, but not limited to, food/pharmaceutical grade anti-foaming agent powder), diluents or fillers (including, but not limited to, water, starch, dextrin, maltodextrin, microcrystalline cellulose, glucose, fructose, sucrose, sorbitol, mannitol, or xylitol; or a combination of any of thereof), gelling agents (including, but not limited to pectin, gelatin, carrageenan, alginic acid, sodium alginate, potassium alginate, ammonium alginate, calcium alginate, starches, maltodextrin, agar, xanthan gum, guar gum, locust bean gum, gellan gum and other natural gums; or a combination of any of thereof), colorants (including, but not limited to, food/pharma grade, natural/artificial color, powder, amaize red (DDW), goji purple (DDW), paprika (DDW; or a combination of any of thereof) and combinations thereof.

### Methods of Treatment

The invention also encompasses methods of treating a subject having a disease or disorder that would benefit from the administration of a cannabinoid, comprising administering to said subject the solid. Preferably, the subject is a human.

In one embodiment, the disease or disorder is selected from: Alzheimer Disease, Amyotrophic Lateral Sclerosis (ALS), pain, anxiety, nausea, vomiting, insomnia, restless leg syndrome (RLS), diabetes mellitus, dystonia, epilepsy, fibromyalgia, gastrointestinal disorders, inflammatory bowel disease, Crohn's disease, irritable bowel syndrome, gliomas, cancer, Hepatitis C, Human Immunodeficiency Virus (HIV) Huntington Disease, hypertension, incontinence, methicillin-resistant Staphyloccus aureus (MRSA), multiple sclerosis, osteoporosis, pruritus, rheumatoid arthritis, insomnia, sleep apnea, or Tourette Syndrome.

In one embodiment, the pain is chronic pain. In another embodiment, the pain is acute pain. In a further embodiment, the acute pain is a migraine. In a further embodiment, the pain is selected from any one or more of the following: post-herpetic neuralgia, trigeminal neuralgia, spinal cord injury pain, carpal tunnel syndrome, phantom limb, ischemic pain, pain resulting from sports injuries, back pain (e.g., low back pain), menstrual pain, gastrointestinal or urethral cramps, skin wounds, burns, or cancer pain. In a preferred embodiment, the pain is cancer pain.

In another embodiment, the nausea and/or vomiting results from a chemotherapy, e.g., cancer chemotherapy. In another embodiment, the nausea and/or vomiting results from opioid use.

In another embodiment, the method is for increasing socialization, increasing relaxation, inducing sleep, reducing the time needed to fall asleep, or for inducing a psychotropic effect (commonly known as a “high”). In another embodiment, the method is for reducing the amount of opioid(s) used by a subject suffering from pain or used by a subject addicted to an opioid.

The composition may be administered once, twice, three, or four times a day, or as needed.

In one embodiment, the invention provides a method of reducing the intensity or duration of pain in a subject (i.e., a subject, e.g., human), in need thereof, comprising the step of administering to the subject an effective amount of a cannabinoid composition of the present invention. In a further embodiment, the method decreases pain intensity in the subject. In a further embodiment, the method decreases pain duration in the subject. In one embodiment, the pain is acute pain. In another embodiment, the pain is chronic pain. In some embodiments, the subject has reduced pain intensity for at least 4 hours, at least 6 hours, at least 8 hours, at least 12 hours, at least 18 hours, or at least 24 hours post administration. In one embodiment, the cannabinoid composition of the present invention has a maximum pain-relieving effect between 1-4 hours or between 1.5-2.5 hours post administration. In another embodiment, the cannabinoid composition of the present invention has an onset of pain-relieving effect within 15 minutes, 20 minutes, 25 minutes, 30 minutes, or within 45 minutes post administration.

In one embodiment, the invention provides a method of reducing or preventing nausea or vomiting in a subject in need thereof, comprising administering to the subject an effective amount of a cannabinoid composition of the present invention. In one embodiment, the nausea or vomiting is opioid induced nausea or vomiting. The opioid inducing the nausea or vomiting may be an opioid analgesic such as hydrocodone, oxycodone, oripavine, dihydromorphine, hydromorphinol, nicomorphine, dipropanoylmorphine, diacetyldihydromorphine, desomorphine, methyldesorphine, heterocodeine, benzylmorphine, dihydroheterocodeine, myrophine, pentamorphone, tramadol, fentanyl, etc. In one embodiment, the cannabinoid composition is administered 0-30 minutes, 30-60 minutes prior to administration of the opioid. In another embodiment, the cannabinoid composition is administered 60 minutes prior to administration of the opioid. In another embodiment, the cannabinoid composition is administered concurrently with the administration of the opioid. In one embodiment, the nausea or vomiting occurs after surgery and results from anesthesia.

In one embodiment, the subject has reduced intensity of nausea in the 2 hours, 3 hours, 4 hours, 5 hours, 6 hours, 8 hours, 12 hours, 18 hours, or 24 hours following initial administration of the cannabinoid containing composition. In one embodiment, the subject has reduced vomiting in the 4 hours, 6 hours, 8 hours, 12 hours, 18 hours, or 24 hours following initial administration of the cannabinoid composition. In one embodiment, the cannabinoid composition of the present invention has a maximum nausea or vomiting reducing effect between 1-4 hours, 1-3 hours, 2-4 hours, or between 1.5-2.5 hours post administration. In another embodiment, the cannabinoid composition of the present invention has an onset of nausea or vomiting reducing effect within 15 minutes, 20 minutes, 25 minutes, 30 minutes, or within 45 minutes post administration.

In one embodiment, the method of reducing nausea or vomiting in a subject includes reducing the occurrence of nausea or vomiting.

The invention is illustrated by the following non-limiting examples.

### EXEMPLIFICATION

### Example 1: Protocol for Manufacture of Self-Emulsifying Oral Cannabinoid Powder

The solid powder compositions described here can be manufactured using a two-step process:

1.1. Preparation of Solution A

The first step involves dissolution of cannabis-derived active ingredient(s) and optional solutes, such as antioxidants, flavorings, etc. in a solvent mixture consisting of surfactant(s), volatile co-solvent(s), and optionally exogeneous oil(s). This solution will generically be termed “Solution A” though its exact composition will vary between examples. When using Method 2.2 and hot extrusion, Solution A can be prepared with or without a co-solvent.

1.2. Preparation of Self-Emulsifying Oral Cannabinoid Powder

After the preparation of Solution A, either one of the two general methods (Method 2.1 or Method 2.2 below) was followed depending on the solubility of the solid carrier in Solution A.

1.2.1. Method 2.1 (when the solid carrier is insoluble in Solution A):

1.2.1.1. Solution A and solid carrier (preferably a highly porous carrier) was mixed(mixing can be accomplished using a V-shaped mixer, ploughshare mixer or a granulator, for example) to coat the surface of the solid carrier.

1.2.1.2. The solvent was evaporated.

1.2.1.3. Passed through an electric sieve to collect powder with desired particle size fraction.

1.2.2. Method 2.2 (when the solid carrier is soluble in Solution A and water or aqueous buffer):

1.2.2.1. Solid carrier was dissolved in Solution A (containing or lacking a co-solvent) to obtain Solution B. Heat can be applied, if necessary.

1.2.2.2. Solution B was dried via hot melt extrusion, spray drying, or lyophilization to form an amorphous film/cylinder/matrix.

1.2.2.3. Using a mill, the amorphous film/cylinder/matrix was broken into particles.

1.2.2.4. The obtained particles were passed through an electric sieve to collect powder with desired particle size fraction.

### Example 2: Preparation of Solution and Manufacture of Self-Emulsifying Oral Cannabinoid Powder

2.1 Preparation of Solution A

Cannabinoid(s) (THC-distillate and/or CBD), terpenes, surfactants (TPGS and polysorbate 80), antioxidants and flavorings were directly added into ethanol while stirring. Gentle heat up to 60° C. was applied, to fully dissolve all ingredients. Stirring was continued until a clear Solution A formed.

2.2 Exemplar Protocols for the Manufacture of Self-Emulsifying Oral Cannabinoid Powder

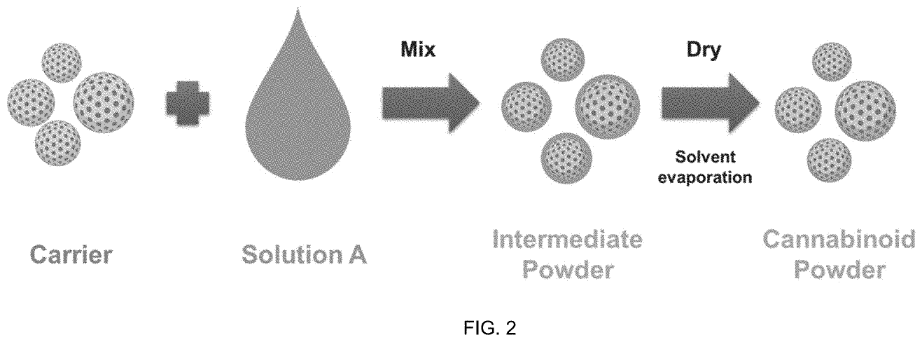

2.2.1. The following provides three exemplary methods for direct coating of solution A on a solid carrier (see FIG. 2, when the carrier is not soluble in Solution A):

2.2.1.1. ⅓-½ of the solid carrier powder was added into a stand mixer with gentle mixing. Solution A was slowly added into the powder. After the liquid formulation was fully “absorbed” and a “moist” powder was formed, the powder was transferred into a V-shaped mixer or ploughshare mixer. The remaining blank (unadsorbed) powder was added and the powder mixture was mixed under middle to high speed until a homogenous powder was formed. The powder was spread on a baking sheet to allow complete evaporation of the ethanol in air at room temperature (16h), in a 40° C. incubation dryer (0.5-1h) or in vacuo at room temperature to 40° C. (2-16h). The dried powder was then passed through a 40-mesh electric sieve to collect desired particle fraction.

2.2.1.2. The solid carrier was added in a high shearing granulator, with the high shearing granulator running at low speed. Solution A was slowly added into the granulator. After loading Solution A, granulation speed was increased and continued for 5-10min until a homogeneous powder was formed. The powder was spread on a baking sheet to allow complete evaporation of ethanol in air at room temperature (16 h), in a 40° C. incubation dryer (0.5-1 h) or in vacuo at room temperature to 40° C. (2-16 h). The dried powder was then passed through a 40-mesh electric sieve to collect desired particle fraction.

2.2.1.3. The solid carrier was added into a fluidized bed machine. Solution A was sprayed to coat the powder and dry in one step. The dried powder was further passed through the 40-mesh electric sieve. The dried powder was passed through a 40-mesh electric sieve to collect desired particle fraction.

2.3.1. For solid carriers that are soluble in Solution A and water or aqueous buffer, see below three exemplar methods:

2.3.1.1. The solid carrier and Solution A were mixed and heated until complete dissolution. The solution was dried via spray drying apparatus and formed a powder. The powder was passed through a 40-mesh electric sieve to collect desired particle fraction.

2.3.1.2. The solid carrier and Solution A was mixed until complete dissolution. The solution was dried via lyophilization apparatus and form “cake collapse”. The “cake collapse” was milled to form powder. The powder was passed through a 40-mesh electric sieve to collect desired particle fraction.

2.3.1.3. Solid carrier and Solution A were mixed and loaded into hot melt extrusion machine to form a cylinder or film. The cylinder or film was cooled and milled into powder. The powder was passed through a 40-mesh electric sieve to collect desired particle fraction.

### Example 3: Effects of Carrier Physical Form on Powder Properties

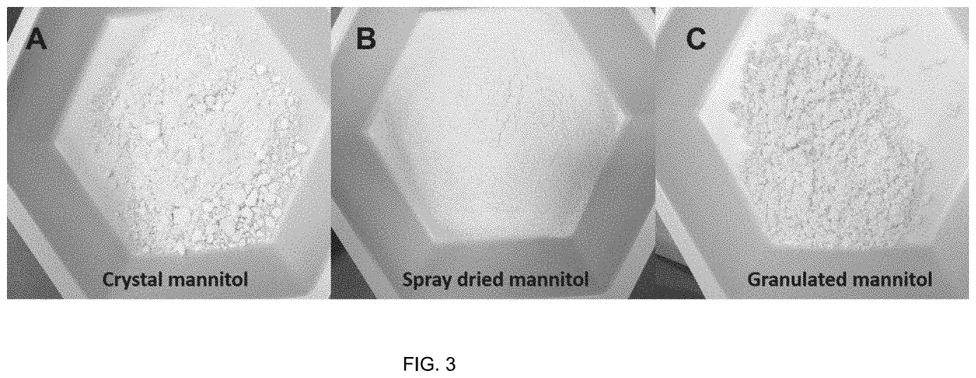

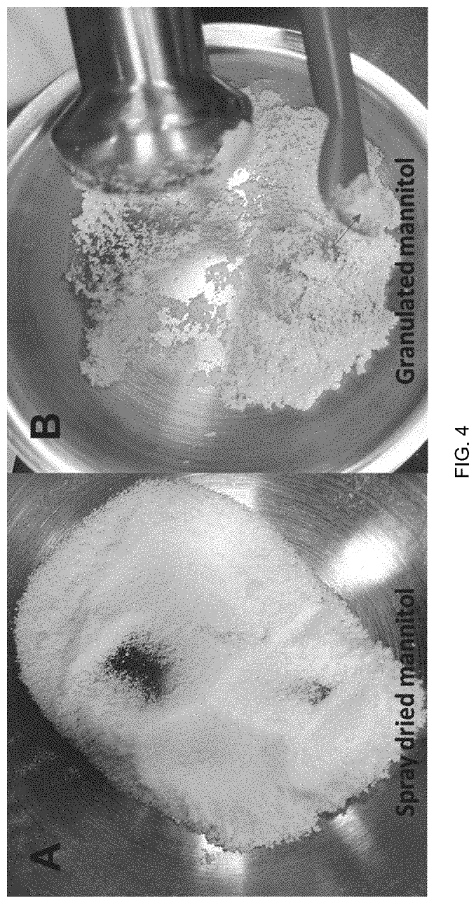

Mannitol was selected as the solid carrier due to its low hygroscopicity compared to other sugars or sugar alcohols offering easy manufacture in solid form and potentially longer shelf life, along with its high, water solubility facilitating fast cannabinoid dissolution kinetics. Possible effects of mannitol as a solid carrier on formulation attributes and processability were investigated. Three different physical forms of mannitol have been evaluated, i.e., crystalline, granulated and spray-dried mannitol. The average particle diameter of spray-dried mannitol was >100 micrometers or preferably around 200 micrometers. Spray dried mannitol performed better than other physical forms; it displayed better flowability vs. the crystalline form due to its spherical particle morphology, and facilitated higher drug loading due to its higher particle surface porosity (FIGS. 3 and 4). Thus, the physical form of the carrier significantly affected both processing and product attributes of oral cannabinoid solids, such as powder characteristics and self-emulsification kinetics.

### Example 4: Enhanced Dissolution of Oral Cannabinoid Powders vs. Liquids

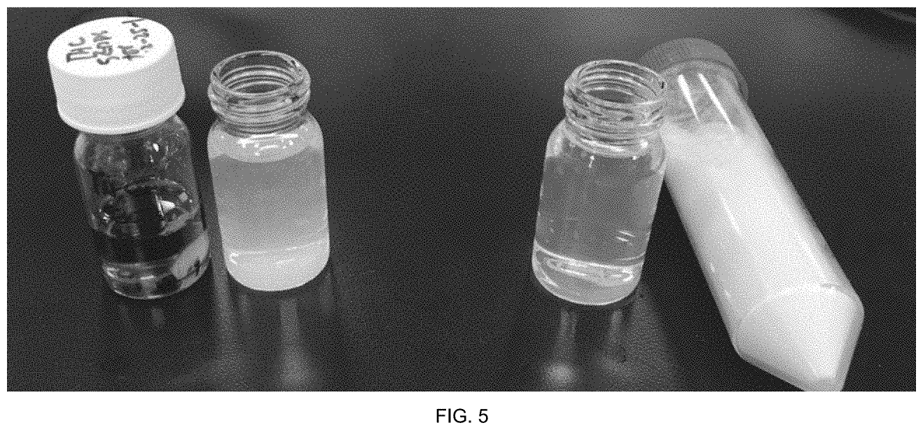

The dispersion time of solid powders utilizing a micron-sized solid carrier was significantly shorter versus that of Solution A intermediate (FIG. 5). This increase in dispersion kinetics was attributed to large surface area provided by the micron-sized solid carrier.

FIG. 5 is a photograph showing the aqueous dispersion properties of THC Solution A (left) vs. THC powder (right). Dispersion time was >5 min. for THC Solution A (self-emulsifying liquid), while THC powder (self-emulsify solid) dissolved within 10 seconds at the same target THC concentration (0.5mg/ml).

### Example 5: Enhanced CBD Dispersion Using Exogeneous Oils

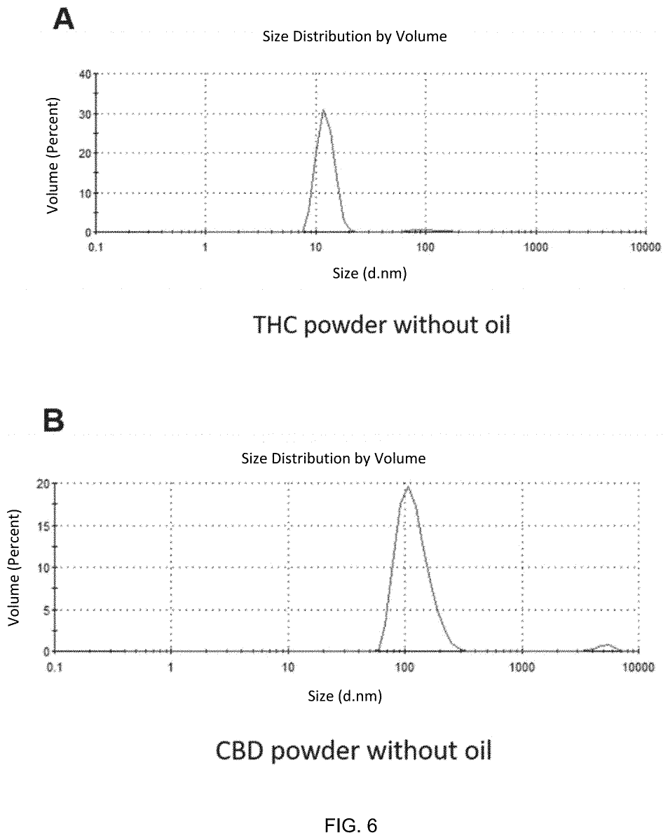

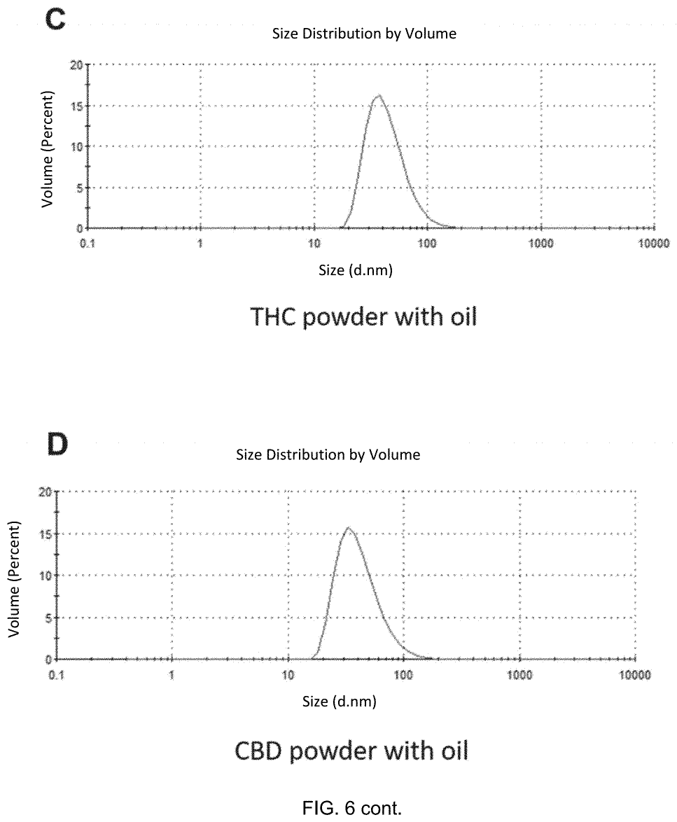

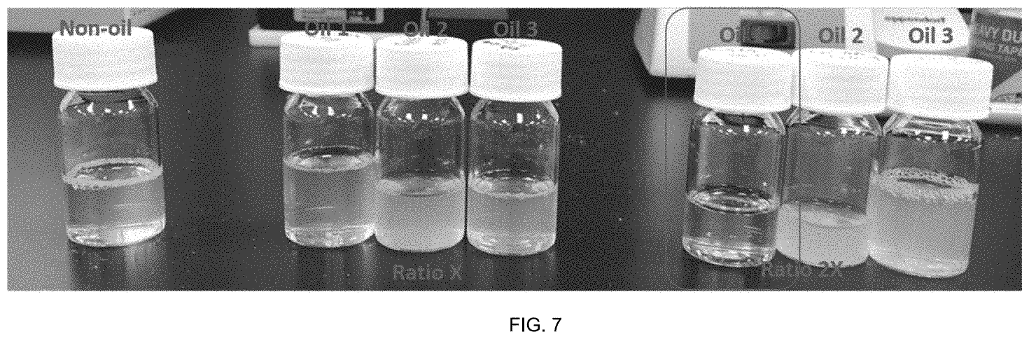

When formulated to self-emulsifying liquids lacking exogeneous oils, we observed that THC could disperse into approximately 10-20 nm micelles in neutral or buffered water. In contrast, the particle size of CBD-equivalent formulations lacking exogeneous oils was significantly larger (approx. 150 nm). Surprisingly, addition of exogenous oils (grapeseed, MCT, and POSAL 53MCT) to THC formulations resulted in an increase in particle size, while that to CBD formulations resulted in a decrease in particle size vs. compositions lacking exogeneous oils. (FIGS. 6 and 7, and Table 2).

TABLE 2
Particle size distribution of aqueous dispersions of CBD powder
formulations. Aqueous dispersions were prepared in DI water
at 0.1 mg/mL CBD. Ratio X is double (2X) ratio 2.

| | | oil: CBD at ratio X | | | oil: CBD at ratio 2X | | |
|---|---|---|---|---|---|---|---|
| Size | Non- | oil | oil | oil | oil | oil | oil |
| by DLS | oil | 1 | 2 | 3 | 1 | 2 | 3 |
| Z- | 147.5 | 157.8 | 295.4 | 193.1 | 53.01 | 433.4 | 217.4 |
| Average | | | | | | | |
| size | | | | | | | |
| (d, nm) | | | | | | | |
| PDI | 0.181 | 0.226 | 0.422 | 0.221 | 0.16 | 0.992 | 0.263 |

### Example 6: Enhancing Product Stability and Prolonging Shelf Life with an Antioxidant

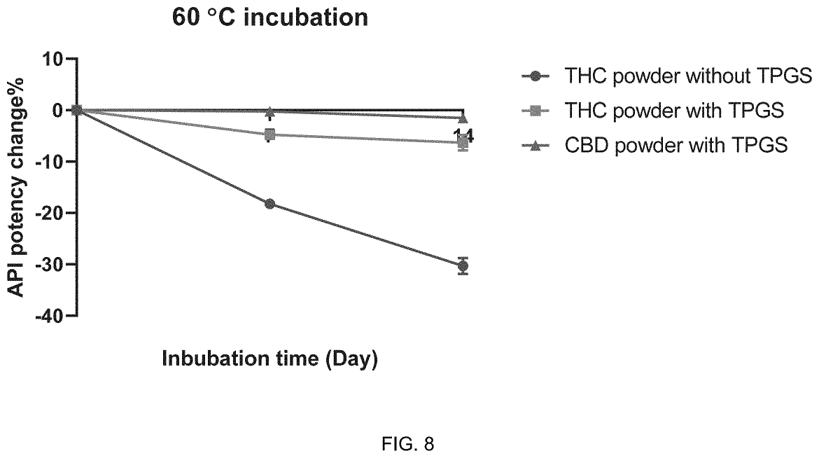

With the high surface area offered by the micron-sized solid powder carrier comes increased potential of exposure to air and chemical degradation of cannabinoid(s) during storage, e.g., through oxidation. This challenge was addressed by incorporating D-α-Tocopherol polyethylene glycol 1000 succinate (TPGS) surfactant into the formulation. TPGS served both as a high HLB surfactant to facilitate micellar dispersion when formulation is diluted in water, and as an antioxidant that significantly improves formulation stability (FIG. 8). A stress test conducted at 60° C. for 14 days (roughly predicting >1 year stability at room temperature, 20-25° C.) indicated that TPGS surfactant can significantly enhance the shelf life of THC powder, presumably offering strong antioxidant effects.

### Example 7: Exemplary Products Containing Self-Emulsifying, Oral Cannabinoid Powders (SCP)

Exemplary products include a cannabinoid beverage additive powder, a cannabinoid effervescent tablet, a hard-gelatin oral cannabinoid capsule, a sublingual cannabinoid tablet, and a coated cannabinoid tablet. These products are described in more detail below:

i. Fast-acting, Cannabinoid Beverage Additive Powder

The composition is described in Tables 3a and 3b below:

TABLE 3a
Composition of fast-acting, cannabinoid beverage additive powder

| | Exemplary excipient(s) (1 | Compositional |
|---|---|---|
| Function | or more, in combination) | range (wt. %) |
| Self- | SCP | 10-80% or 10-60% |
| emulsifying | | or 20-60% or 30-60% |
| cannabinoid | | or 35-55% or 40-50% |
| powder (SCP) | | |
| Diluent, | Starch, dextrin, maltodextrin, | 0-70% or 10 to |
| filler | microcrystalline cellulose, | 60% or 20 to 60% |
| | sucralose, glucose, fructose, | or 35 to 60% |
| | sucrose, sorbitol, mannitol, | |
| | xylitol; or any combination | |
| | thereof | |
| Flavorant | Potassium, advantame, | 0.1-50% or 0.1-25% |
| | aspartame, neotame, | |
| | saccharin, mannitol, Luo Han | |
| | Guo (monk) fruit extract | |
| | powder, sucralose, stevia, | |
| | glucose, fructose, dextrose, | |
| | sucrose, sorbitol, xylitol, or | |
| | any combination thereof | |
| Flavorant | Food/pharma grade, natural/ | 0-25% |
| | artificial flavor, powder, | |
| | or any combination thereof | |
| Colorant | Food/pharma grade, natural/ | 0-10% |
| | artificial color, powder, | |
| | or any combination thereof | |
| Acidifier | Citric acid, malic acid, | 0-10% or 0-5% |
| | tartaric acid, fumaric acid, | |
| | adipic acid, boric acid, or any | |
| | combination thereof | |

TABLE 3b
Composition of fast-acting, cannabinoid beverage powder

| | | Compositional |
|---|---|---|
| Function | Exemplary excipient(s) | range (wt. %) |
| Self- | SCP | 30-60% or 35-55% |
| emulsifying | | or 40-50% |
| cannabinoid | | |
| powder (SCP) | | |
| Diluent, | Mannitol, glucose, sucrose, | 20 to 60% or |
| filler | fructose, sorbitol, xylitol | 35 to 60% |
| Flavorant | Potassium, advantame, | 0.1-50% |
| | aspartame, neotame, | |
| | saccharin, mannitol, Luo Han | |
| | Guo (monk) fruit extract | |
| | powder, sucralose, stevia, | |
| | glucose, fructose, dextrose, | |
| | sucrose, sorbitol, xylitol; or | |
| | any combination thereof | |
| Flavorant | Food/pharma grade, natural/ | 0-25% |
| | artificial flavor, powder, | |
| | or any combination thereof | |
| Colorant | Food/pharma grade, natural/ | 0-10% |
| | artificial color, powder, | |
| | or any combination thereof | |
| Acidifier | Citric acid, malic acid, | 0-10% or 0-5% |
| | tartaric acid, fumaric acid, | |
| | adipic acid, boric acid, or any | |
| | combination thereof | |

The observed product attributes for the fast-acting, cannabinoid beverage additive powder are described in Table 4 below:

TABLE 4
Key product attributes of fast-acting,
cannabinoid beverage additive powder

| | Fast-acting, flavored | |
|---|---|---|
| | beverage additive, powder; | |
| Dosage form; units: | 5 mg/g cannabinoid | |
| Dissolution time in water | <1 | min |
| Appearance of aqueous dispersion | Translucent solution | |
| Particle size of aqueous dispersion | <150 | nm |
| (Z-average, diameter) | | |

| | |
|---|---|
| Organoleptic properties of aqueous | Minimal/no discernible bitterness |
| dispersion | |
| Shelf-life, stability | >6 months at ambient |
| | temperature (20-25° C.) |

ii. Fast-Acting, Cannabinoid Effervescent Tablet

The composition is described in Tables 5a and 5b below:

TABLE 5a
Composition of fast-acting, cannabinoid effervescent tablet

| | Exemplary excipient(s) (1 | Compositional |
|---|---|---|
| Function | or more, in combination) | range (wt. %) |
| Effervescent | Sodium/potassium/magnesium/ | 15-60% or 20 to 40% |
| | calcium carbonate, | |
| | sodium/potassium/magnesium/ | |
| | calcium bicarbonate, or any | |
| | combination thereof | |
| Self- | SCP | 10-50% or 15 to 45% |
| emulsifying | | or 20 to 40% |
| cannabinoid | | |
| powder (SCP) | | |
| Acidifier | Citric acid, malic acid, | 15-60% or 20-50% |
| | tartaric acid, fumaric acid, | |
| | adipic acid, boric acid, or any | |
| | combination thereof | |
| Lubricant | PEG (3,000Da to 35,000Da), | 0.1-20% or 0.1-10% |
| | magnesium stearate, sodium | |
| | stearyl fumarate, or | |
| | any combination thereof | |
| Flavorant | Food/pharma grade, natural/ | 0.1-10% |
| | artificial flavor, powder, | |
| | or any combination thereof | |
| Colorant | Food/pharma grade, natural/ | 0.1-10% |
| | artificial color, powder, | |
| | or any combination thereof | |
| Sweetener | Potassium, advantame, | 0.1-50% |
| | aspartame, neotame, | |
| | saccharin, mannitol, Luo Han | |
| | Guo (monk) fruit extract | |
| | powder, sucralose, stevia, | |
| | glucose, fructose, | |
| | sucrose, sorbitol, xylitol, | |
| | or any combination thereof | |
| Diluent, | Starch, dextrin, maltodextrin, | 0-70% |
| filler | microcrystalline cellulose, | |
| | glucose, fructose, sucrose, | |
| | sorbitol, mannitol, xylitol, | |
| | or any combination thereof | |
| Defoamer | Food/pharma grade anti-foaming | 0.1-10% |
| | agent, powder | |
| Flavorant | Sodium chloride | 0.1-5% |

TABLE 5b
Composition of fast-acting, cannabinoid effervescent tablet

| | | Compositional |
|---|---|---|
| Function | Excipient | range (wt. %) |
| Effervescent | Sodium bicarbonate | 20 to 40% |
| Self- | SCP | 15 to 45% or 20 to 40% |
| emulsifying | | |
| cannabinoid | | |
| powder (SCP) | | |
| Acidifier | Citric acid | 15-60% or 20-50% |
| Lubricant | PEG 8000 | 0.1-20% or 0.1-10% |
| Flavorant | Food/pharma grade, natural/ | 0.1-10% |
| | artificial flavor, powder, | |
| | or any combination thereof | |
| Colorant | Food/pharma grade, natural/ | 0.1-10% |
| | artificial color, powder, | |
| | or any combination thereof | |
| Sweetener | Potassium, advantame, | 0.1-50% |
| | aspartame, neotame, | |
| | saccharin, mannitol, Luo Han | |
| | Guo (monk) fruit extract | |
| | powder, sucralose, stevia, | |
| | glucose, fructose, | |
| | sucrose, sorbitol, xylitol, | |
| | or any combination thereof | |
| Diluent, | Mannitol | 0-40% |
| filler | | |
| Defoamer | Simethicone | 0.1-10% |
| Flavorant | Sodium chloride | 0.1-5% |

The observed product attributes for the fast-acting, cannabinoid effervescent tablet are described in Table 6 below:

TABLE 6
Key product attributes of fast-acting,
cannabinoid effervescent tablet

| | Fast-acting, effervescent | |
|---|---|---|
| Dosage form; units | tablet; 2.5 mg/10 mg cannabinoid | |
| Dissolution time in water | <5 | min |
| Appearance of aqueous dispersion | Translucent solution | |
| Particle size of aqueous dispersion | <200 | nm |
| (Z-average, diameter) | | |

| | |
|---|---|
| Organoleptic properties of aqueous | Minimal/no discernible bitterness |
| dispersion | |

iii. Fast-Acting, Hard-Gelatin, Oral Cannabinoid Capsule

The composition is described in Table 7 below:

TABLE 7
Composition of fast-acting, hard-gelatin oral capsule

| | | Compositional |
|---|---|---|
| Function | Exemplary excipient(s) | range (wt. %) |
| Diluent, | Starch, dextrin, maltodextrin, | 0-70% |
| filler | microcrystalline cellulose, | |
| | glucose, fructose, sucrose, | |
| | sorbitol, mannitol, xylitol, | |
| | or any combination thereof | |
| Self- | SCP | 0.1-99.9% |
| emulsifying | | |
| cannabinoid | | |
| powder (SCP) | | |
| Hard capsule | Gelatin, vegan or vegetarian | 0.1 to 99.9% |
| | hard capsule, size 000, 00, | |
| | 0, 1, 2, 3, 4 or 5 | |
| | Enteric-coated gelatin, vegan | |
| | or vegetarian hard capsule, size | |
| | 000, 00, 0, 1, 2, 3, 4 or 5 | |

The key product attributes of the composition are described in Table 8 below:

TABLE 8
Key product attributes of fast-acting,
hard-gelatin oral cannabinoid capsule

| | Fast-acting, hard-gelation |
|---|---|
| Dosage form; units: | oral capsule; 5 mg cannabinoid |
| Dissolution time in simulated | <20 min |
| gastric fluid at 37° C.: | |
| Appearance of aqueous dispersion: | Translucent solution |

iv. Fast-Acting, Sublingual Cannabinoid Tablet

The composition of the tablet is described below in Table 9:

TABLE 9
Composition of fast-acting, sublingual cannabinoid tablet

| | Exemplary excipients (1 | Compositional |
|---|---|---|
| Function | or more, in combination) | range (wt. %) |
| Diluent, | Starch, dextrin, maltodextrin, | 0-70% |
| filler | microcrystalline cellulose, | |
| | glucose, fructose, sucrose, | |
| | sorbitol, mannitol, xylitol, | |
| | or any combination thereof | |
| Self- | SCP | 10-50% |
| emulsifying | | |
| cannabinoid | | |
| powder (SCP) | | |
| Flavorant | Potassium, advantame, | 0.1-50% |
| | aspartame, neotame, | |
| | saccharin, mannitol, Luo Han | |
| | Guo (monk) fruit extract | |
| | powder, sucralose, stevia, | |
| | glucose, fructose, | |
| | sucrose, sorbitol, xylitol, | |
| | or any combination thereof | |
| Flavorant | Food/pharma grade, natural/ | 0-20% |
| | artificial flavor, bitterness | |
| | modifier (FONA) powder, or | |
| | any combination thereof” | |
| Lubricant | PEG (3,000Da to 35,000Da), | 0.1-20% |
| | magnesium stearate, sodium | |
| | stearyl fumarate, or | |
| | any combination thereof” | |
| Colorant | Food/pharma grade, natural/ | 0-10% |
| | artificial color, powder, | |
| | or any combination thereof | |
| Acidifier | Citric acid, malic acid, | 0-10% |
| | tartaric acid, fumaric acid, | |
| | adipic acid, boric acid, or any | |
| | combination thereof | |

v. Coated Cannabinoid Tablet

The composition of the tablet is described in Table 10 below:

TABLE 10
Composition of coated cannabinoid tablet

| | Exemplary excipients (1 | Compositional |
|---|---|---|
| Function | or more, in combination) | range (wt. %) |
| Diluent, | Starch, dextrin, maltodextrin, | 0-70% |
| filler | microcrystalline cellulose, | |
| | glucose, fructose, sucrose, | |
| | sorbitol, mannitol, xylitol, | |
| | or any combination thereof | |
| Self- | SCP | 10-99% |
| emulsifying | | |
| cannabinoid | | |
| powder (SCP) | | |
| Lubricant | PEG (3,000Da to 35,000Da), | 0.1-20% |
| | magnesium stearate, sodium | |
| | stearyl fumarate, or | |
| | any combination thereof | |
| Coating | Polymethacrylates (see Table 11 | 0.1-10% |
| | for chemical/trade names): | |
| | Eudragit E series (fast-acting); | |
| | Eudragit L series, Eudragit S | |
| | series, Eudragit FS series, | |
| | Eastacryl 30D, Kollicoat MAE 30 | |
| | DP, Kolllicoat MAE 100 P, | |
| | Acryl-EZE, Acryl-EZE 93 A, | |
| | Acryl-EZE MP (enteric coating); | |
| | Eudragit RL series, Eudragit RS | |
| | series, Eudragit NE 30D, Eudragit | |
| | NE 40D, and Eudragit NM30D | |
| | (delayed/sustained release), or | |
| | any combination thereof | |

TABLE 11
Chemical name and CAS registry number of polymethacrylates (Rowe,
R. C., P. Sheskey, and M. Quinn, Handbook of pharmaceutical excipients.
2009: Libros Digitales-Pharmaceutical Press).

| Chemical name | Trade name | Company name | CAS number |
|---|---|---|---|
| Poly(butyl methacrylate, (2- | Eudragit E 100 | Evonik Industries | 24938-16-7 |
| dimethylaminoethyl) methacrylate, | Eudragit E 12.5 | Evonik Industries | |
| methyl methacrylate) 1:2:1 | Eudragit E PO | Evonik Industries | |
| Poly(ethyl acrylate, methyl | Eudragit NE 30 D | Evonik Industries | 9010-88-2 |
| methacrylate) 2:1 | Eudragit NE 40 D | Evonik Industries | |
| | Eudragit NM 30 D | Evonik Industries | |
| Poly(methacrylic acid, methyl | Eudragit L 100 | Evonik Industries | 25806-15-1 |
| methacrylate) 1:1 | Eudragit L 12.5 | Evonik Industries | |
| | Eudragit L 12.5 P | Evonik Industries | |
| Poly(methacrylic acid, ethyl | Acryl-EZE | Colorcon | 25212-88-8 |
| acrylate) 1:1 | Acryl-EZE 93A | Colorcon | |
| | Acryl-EZE MP | Colorcon | |
| | Eudragit L 30 D-55 | Evonik Industries | |
| | Eudragit L 100-55 | Evonik Industries | |
| | Eastacryl 30D | Eastman Chemical | |
| | Kollicoat MAE 30 DP | BASF Fine Chemicals | |
| | Kollicoat MAE 100 P | BASF Fine Chemicals | |
| Poly(methacrylic acid, methyl | Eudragit S 100 | Evonik Industries | 25086-15-1 |
| methacrylate) 1:2 | Eudragit S 12.5 | Evonik Industries | |
| | Eudragit S 12.5 P | Evonik Industries | |
| Poly(methyl acrylate, methyl | Eudragit FS 30D | Evonik Industries | 26936-24-3 |
| methacrylate, methacrylic acid) 7:3:1 | | | |
| Poly(ethyl acrylate, methyl | Eudragit RL 100 | Evonik Industries | 33434-24-1 |
| methacrylate, | Eudragit RL PO | Evonik Industries | |
| trimethylammonioethyl | Eudragit RL 30 D | Evonik Industries | |
| methacrylate chloride) 1:2:0.2 | Eudragit RL 12.5 | Evonik Industries | |
| Poly(ethyl acrylate, methyl | Eudragit RS 100 | Evonik Industries | 33434-24-1 |
| methacrylate, | Eudragit RS PO | Evonik Industries | |
| trimethylammonioethyl | Eudragit RS 30 D | Evonik Industries | |
| methacrylate chloride) 1:2:0.1 | Eudragit RS 12.5 | Evonik Industries | |

vi. Fast-Acting, Cannabinoid Gummy

The composition of the gummy is described below in Table 12:

TABLE 12
Composition of fast-acting cannabinoid gummy

| | Exemplary excipients (1 | Compositional |
|---|---|---|
| Function | or more, in combination) | range (wt. %) |
| Coating | Starch, dextrin, maltodextrin, | 0-20% |
| | microcrystalline cellulose, | |
| | acesulfame potassium, aspartame, | |
| | neotame, saccharin, mannitol, Luo | |
| | Han Guo (monk) fruit extract powder, | |
| | sucralose, stevia, glucose, fructose, | |
| | sucrose, sorbitol, or xylitol, citric | |
| | acid, malic acid, tartaric acid, | |
| | fumaric acid, adipic acid, boric | |
| | acid, or any combination thereof | |
| Gelling agent | Pectin, gelatin, carrageenan, alginic | 1-20% |
| | acid, sodium alginate, potassium | |
| | alginate, ammonium alginate, calcium | |
| | alginate, starches, maltodextrin, agar, | |
| | xanthan gum, guar gum, locust bean | |
| | gum, gellan gum and other natural | |
| | gums, or any combination thereof | |
| Diluent | Water | 5-50% |
| Cannabinoid | Cannabinoid | 0-20% |
| Oil phase | An oil comprising an MCT and/or | 0-30% |
| | LCT can be selected from the group | |
| | consisting of borage oil, castor oil, | |
| | coconut oil, cottonseed oil, soybean | |
| | oil, safflower oil, sunflower oil, | |
| | castor oil, corn oil, olive oil, palm | |
| | oil, almond oil, grapeseed oil, palm | |
| | kernel oil, peanut oil, poppy seed | |
| | oil, canola oil, hydrogenated soybean | |
| | oil, hydrogenated vegetable oils, | |
| | sesame oil, triolein, trilinolein, and | |
| | trilinolenin, or any combination thereof | |
| Self- | SCP | 0.01-20% |
| emulsifying | | |
| cannabinoid | | |
| powder (SCP) | | |
| Flavorant | Acesulfame potassium, aspartame, | 0.1-50% |
| | neotame, saccharin, mannitol, Luo Han | |
| | Guo (monk) fruit extract powder, | |
| | sucralose, stevia, glucose, fructose, | |
| | sucrose, sorbitol, xylitol, or any | |
| | combination thereof | |
| Flavorant | Food/pharma grade, natural/ | 0-20% |
| | artificial flavor, bitterness | |
| | modifier | |
| Flavorant | Maple syrup, glucose syrup, | 10-50% |
| | tapioca syrup, corn syrup, high | |
| | fructose corn syrup, golden | |
| | syrup, cane syrup, agave syrup, | |
| | or any combination thereof | |
| Colorant | Food/pharma grade, natural/ | 0-15% |
| | artificial color | |
| Acidifier | Citric acid, malic acid, | 0-10% |
| | tartaric acid, fumaric acid, | |
| | adipic acid, boric acid, | |
| | sodium citrate, or any | |
| | combination thereof | |

vii. Fast-Acting, Cannabinoid Effervescent Powder Mix

The composition of the mix is described below in Table 13:

TABLE 13
Composition of fast-acting, cannabinoid effervescent powder mix

| | Exemplary excipient(s) (1 | Compositional |
|---|---|---|
| Function | or more, in combination) | range (wt. %) |
| Effervescent | Sodium/potassium/magnesium/ | 15-60% |
| | calcium carbonate, | |
| | sodium/potassium/magnesium/ | |
| | calcium bicarbonate, or any | |
| | combination thereof | |
| Self- | SCP | 10-50% |
| emulsifying | | |
| cannabinoid | | |
| powder (SCP) | | |
| Acidifier | Citric acid, malic acid, | 15-60% |
| | tartaric acid, fumaric acid, | |
| | adipic acid, boric acid, or any | |
| | combination thereof | |
| Flavorant | Food/pharma grade, natural/ | 0.1-10% |
| | artificial flavor, powder, | |
| | or any combination thereof | |
| Colorant | Food/pharma grade, natural/ | 0.1-10% |
| | artificial color, powder, | |
| | or any combination thereof | |
| Sweetener | Acesulfame potassium, aspartame, | 0.1-50% |
| | neotame, saccharin, mannitol, Luo Han | |
| | Guo (monk) fruit extract powder, | |
| | sucralose, stevia, glucose, fructose, | |
| | sucrose, sorbitol, xylitol, or any | |
| | combination thereof | |
| Diluent, | Starch, dextrin, maltodextrin, | 0-70% |
| filler | microcrystalline cellulose, | |
| | glucose, fructose, sucrose, | |
| | sorbitol, mannitol, xylitol, | |
| | or any combination thereof | |
| Defoamer | Food/pharma grade anti-foaming | 0.1-10% |
| | agent powder, or any combination | |
| | thereof | |

### REFERENCES

1. Nikolakakis, I. and I. J. P. Partheniadis, Self-emulsifying granules and pellets: Composition and formation mechanisms for instant or controlled release. 2017. 9(4): p. 50.
2. Gupta, S., R. Kesarla, and A. J. I. p. Omri, Formulation strategies to improve the bioavailability of poorly absorbed drugs with special emphasis on self-emulsifying systems. 2013. 2013.
3. Goldstein, J. H., et al., Homogenous cannabis compositions and methods of making the same. 2017, Google Patents.
4. Rowe, R. C., P. Sheskey, and M. Quinn, Handbook of pharmaceutical excipients. 2009: Libros Digitales-Pharmaceutical Press.

All references, articles, patent applications, patent publications and patents are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

1. A self-emulsifying, solid composition comprising a population of particles, wherein each particle comprises: i) at least one of a cannabinoid and a terpene, ii) at least one surfactant having an HLB value greater than 10, and iii) a solid carrier in powder or granular form,
wherein the solid carrier is a water-soluble solid carrier or a water-insoluble solid carrier;
wherein when the solid carrier a water-soluble solid carrier, then the surfactant and the cannabinoid or the terpene are present on the surface of the solid carrier or the water-soluble solid carrier, surfactant, and the cannabinoid or the terpene form an amorphous matrix; and
wherein when the solid carrier is a water-insoluble solid carrier, then the surfactant and the cannabinoid or the terpene are present on the surface of the solid carrier.

2. The composition of claim 1, wherein the solid carrier is a water-soluble solid carrier and the surfactant and the cannabinoid or the terpene are present on the surface of the solid carrier.

3. The composition of claim 1, wherein the solid carrier is a water-soluble solid carrier and the water-soluble solid carrier, surfactant, and the cannabinoid or the terpene form an amorphous matrix.

4. The composition of claim 1, wherein the solid carrier is a water-insoluble solid carrier, then the surfactant and the cannabinoid or the terpene are present on the surface of the solid carrier.

5. The composition of claim 1, wherein the composition comprises a first population of particles and a second population of particles wherein:
a. each particle of the first population comprises: i) at least one of a cannabinoid and a terpene, ii) at least one surfactant having an HLB value greater than 10, and iii) a water-soluble solid carrier in powder or granular form; and
b. each particle of the second population comprises i) at least one of a cannabinoid and a terpene, ii) at least one surfactant having an HLB value greater than 10, and iii) a water-insoluble solid carrier in powder or granular form.

6. The composition of claim 1, wherein each particle comprises a cannabinoid and a terpene.

7. The composition of claim 1, wherein each particle comprises a cannabinoid.

8. The composition of claim 1, wherein each particle comprises a terpene.

9. The composition of claim 7, wherein the cannabinoid is selected from the group consisting of CBD, Δ9-tetrahydrocannabinol (Δ9-THC), Δ8-tetrahydrocannabinol (Δ8-THC), THCA, THCB, THCP, THCV, CBD, CBDA, CBDB, CBDP, CBDV, CBDL, CBC, CBCA, CBCV, CBCN, CBV, CBG, CBGA, CBGV, CBN, CBL, and CBE, or a combination thereof.

10. The composition of claim 9, wherein the cannabinoid is Δ9-THC or CBD, or a combination thereof.

11. (canceled)

12. The composition of claim 1, wherein the average diameter of the solid carrier is less than about 3000 micrometers.

13. The composition of claim 12, wherein the average diameter of the solid carrier is greater than 10 micrometers.

14. The composition of claim 1, wherein the solid carrier is a water-soluble solid carrier.

15. The composition of claim 14, wherein the solid carrier is selected from the group consisting of sucrose, fructose, maltose, glucose, lactose, galactose, mannitol, sorbitol, xylitol, starch, dextrin, maltodextrin, hydroxypropyl-methylcellulose, and sodium carboxymethyl starch.

16. The composition of claim 14, wherein the solid carrier is a sugar or a sugar-alcohol.

17. (canceled)

18. The composition of claim 16, wherein the sugar alcohol is mannitol.

19. The composition of claim 18, wherein the mannitol is crystalline, granulated, or spray-dried.

20. The composition of claim 19, wherein the mannitol is spray-dried mannitol.

21. The composition of claim 1, wherein the amount of the cannabinoid or the terpene is between about 0.01 and about 20% w/w.

22. (canceled)

23. The composition of claim 1, wherein the surfactant is selected from the group consisting of D-α-tocopherol polyethylene glycol 1000 succinate (TPGS), polysorbates, PEG-castor oils, phospholipid, lauroyl-L-carnitine, and poloxamers.

24. (canceled)

25. (canceled)

26. The composition of claim 1, wherein the surfactant has antioxidant properties.

27. The composition of claim 1, wherein the amount of the surfactant is between about 0.1 to about 50% w/w.

28. The composition of claim 1, wherein the amount of the solid carrier is between about 50 to about 99% w/w.

29. The composition of claim 1, wherein the composition does not comprise a co-solvent.

30. The composition of claim 1, further comprising one or more of an exogenous oil, an antioxidant, and a flavoring.

31-35. (canceled)

36. The composition of claim 1, wherein the cannabinoid is THC and the composition does not comprise an exogenous oil.

37. The composition of claim 1, wherein the cannabinoid is CBD and the composition further comprises an exogenous oil.

38-42. (canceled)

43. The composition of claim 1, wherein the composition is further formulated as a tablet or capsule.

44. The composition of claim 1, wherein the composition forms self-assembled nanoparticles upon addition to an aqueous medium.

45. The composition of claim 44, wherein the nanoparticles are about 10 to about 800 nm in diameter.

46. The composition of claim 1, wherein the solid composition dissolves within about one minute of addition to an aqueous medium to form a solution, and wherein the solution comprises at least about 0.01 mg/ml cannabinoid.

47-49. (canceled)

50. The composition of claim 1, wherein the solid carrier is a water-insoluble solid carrier.

51. The composition of claim 50, wherein the water-insoluble solid carrier is selected from the group consisting microcrystalline cellulose, silicon dioxide, magnesium aluminometasilicate, silica, sucrose monopalmitate, and calcium silicate.

52. The composition of claim 50, wherein the water-insoluble solid carrier is present in an amount between about 50 and about 99% w/w.

53. The composition of claim 5, wherein the water-soluble solid carrier is a sugar or a sugar alcohol.

54. The composition of claim 5, wherein the water-insoluble solid carrier is selected from the group consisting microcrystalline cellulose, silicon dioxide, magnesium aluminometasilicate, silica, sucrose monopalmitate, and calcium silicate.

55-57. (canceled)

58. A method for the preparation of a self-emulsifying, solid composition comprising a population of particles, wherein each particle comprises i) at least one of a cannabinoid or a terpene, ii) at least one surfactant having an HLB value greater than 10, and iii) a solid carrier in powder or granular form, wherein the solid carrier is a water-soluble carrier or a water-insoluble carrier, and wherein the surfactant and the cannabinoid or the terpene are present on the surface of the solid carrier, the method comprising:
a. dissolving the cannabinoid or the terpene in a solvent mixture to form a first solution, wherein the solvent mixture comprises a volatile solvent and the surfactant;
b. mixing the solid carrier with the first solution to form a mixture, wherein the solid carrier is insoluble in the first solution;
c. evaporating the solvent from the mixture; and
d. collecting the particles.

59-67. (canceled)

68. A method for the preparation of a self-emulsifying, solid composition comprising a population of particles, wherein each particle comprises i) at least one of a cannabinoid or a terpene, ii) at least one surfactant having an HLB value greater than 10, and iii) a water-soluble solid carrier in powder or granular form, wherein the solid carrier, surfactant and the cannabinoid or the terpene form an amorphous solid, the method comprising:
a. dissolving the cannabinoid or the terpene in a solvent mixture to form a first solution, wherein the solvent mixture comprises a volatile solvent, and the surfactant;
b. mixing the water-soluble, solid carrier with the first solution to form a second solution, wherein the water-soluble, solid carrier is soluble in the first solution;
c. drying the second solution to form an amorphous solid;
d. breaking the amorphous solid into particles; and
e. collecting the particles.

69-81. (canceled)

82. A method for the preparation of a self-emulsifying, solid composition comprising a population of particles, wherein each particle comprises i) at least one of a cannabinoid or a terpene, ii) at least one surfactant having an HLB value greater than 10, and iii) a water-soluble solid carrier in powder or granular form, wherein the solid carrier, surfactant and the cannabinoid or the terpene form an amorphous solid, the method comprising:
a. mixing the cannabinoid or the terpene with the water-soluble solid carrier and forming an amorphous matrix by hot melt extrusion;
b. breaking the amorphous solid into particles; and
c. collecting the particles.

83-96. (canceled)
